(19) United States
(12) Patent Application Publication
(10) Pub. No.: US 2023/0293529 A1
(43) Pub. Date: Sep. 21, 2023

(54) DOSING REGIMENS FOR N-((5-FLUORO-2,3-DIHYDROBENZOFURAN-4-YL)METHYL)-8-(2-METHYLPYRIDIN-3-YL)-[1,2,4]TRIAZOLO[4,3-C]PYRIMIDIN-5-AMINE, OR A PHARMACEUTICALLY ACCEPTABLE SALT THEREOF, FOR USE IN TREATING PRC2-MEDIATED DISEASES OR DISORDERS

(71) Applicant: Novartis AG, Basel (CH)

(72) Inventors: Yi GU, Brookline, MA (US); Sebastien JEAY, Niffer (FR); Yi JIN, Stein (CH); Marc LAISNEY, Mulhouse (FR); Christophe MEILLE, Michelbach Ie haut (FR); Prakash Dahyabhai MISTRY, Surrey (GB); Jonathan Guy MOGGS, Leymen (FR); Andreas WEISS, Zurich, (CH); Mélanie Monique Laura WILBAUX, Hagenthal le Bas (FR)

(21) Appl. No.: 17/999,694

(22) PCT Filed: May 25, 2021
(86) PCT No.: PCT/IB2021/054555

§ 371 Date: Nov. 22, 2022

(30) Foreign Application Priority Data
May 28, 2020 (WO) PCT/CN2020/092796

(51) Int. Cl. A61K 31/519; A61P 35/00

(52) U.S. Cl. CPC A61K 31/519; A61P 35/00

(57) ABSTRACT

The invention provided herein provides dosage and dosage regimens for N-((5-fluoro-2,3-dihydrobenzofuran-4-yl)methyl)-8-(2-methylpyridin-3-yl)-[1,2,4]triazolo[4,3-c]pyrimidin-5-amine (Compound (1)), or a pharmaceutically acceptable salt thereof, Compound (1) for the treatment of PRC2-mediated diseases or disorders. In addition, the invention provides using such dosage and dosage regimens in methods for treating PRC2-mediated diseases or disorders.

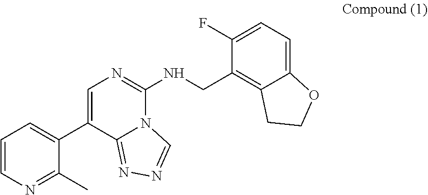 [SMILES: Cc1ncccc1-c1cnc(NCc2c(F)ccc3c2CCO3)n2cnnc12]

### FIELD OF THE INVENTION

This invention relates to dosing regimens for N-((5-Fluoro-2,3-dihydrobenzofuran-4-yl)methyl)-8-(2-methyl pyridin-3-yl)-[1,2,4]triazolo[4,3-c]pyrimidin-5-amine, or a pharmaceutically acceptable salt thereof, for the treatment of Polycomb Repressive Complex 2 (PRC2)-mediated diseases or disorders. The invention also relates to methods of treatment of Polycomb Repressive Complex 2 (PRC2)-mediated diseases or disorders using such dosing regimens.

### BACKGROUND OF THE INVENTION

Polycomb group (PcG) proteins are chromatin modifying enzymes that are dysregulated in many human cancers. The Polycomb Repressive Complex 2 (PRC2), which includes SUZ12 (suppressor of zeste 12), EED (embryonic ectoderm development) and the catalytic subunit, EZH2 (enhancer of zeste homolog 2), represses genes by methylating the core histone H3 lysine 27 (H3K27me3) at and around the promoter regions of target genes. PRC2 is the critical component of cellular machinery involved in the epigenetic regulation of gene transcription and plays critical function in development and tissue differentiation and regeneration. Although EZH2 is the catalytic subunit, PRC2 requires at least EED and SUZ12 for its methyltransferase activity. EED, SUZ12 and EZH2 are overexpressed in many cancers, including but not limited to breast cancer, prostate cancer, hepatocellular carcinoma and etc. EZH2 activating mutations have been identified in DLBCL (diffused large B cell lymphoma) patients and FL (follicular lymphoma) patients. Inhibition of PRC2 methyltransferase activity by compounds competing with the cofactor S-adenosyl methionine (SAM) in DLBCL reverses H3K27 methylation, re-activates expression of target genes and inhibits tumor growth/proliferation. Therefore, PRC2 provides a pharmacological target for DLBCL and other cancers.

### SUMMARY OF THE INVENTION

There remains a need for new treatments and therapies for PRC2-mediated diseases or disorders. International patent application WO2016/103155A1 discloses triazolopyrimidine derivatives and their use in treating PRC2-mediated diseases or disorders, however there is no disclosure regarding a dosing regimen. Accordingly, the invention provides dosages, dosage forms, medicaments, compositions and dosing regimens for N-((5-fluoro-2,3-dihydrobenzofuran-4-yl)methyl)-8-(2-methylpyridin-3-yl)-[1,2,4]triazolo[4,3-c]pyrimidin-5-amine (Compound 1), or a pharmaceutically acceptable salt thereof, for use in treating PRC2-mediated diseases or disorders. N-((5-fluoro-2,3-dihydrobenzofuran-4-yl)methyl)-8-(2-methylpyridin-3-yl)-[1,2,4]triazolo[4,3-c]pyrimidin-5-amine (Compound 1) has the following structure:

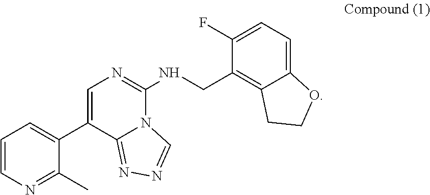 [SMILES: Cc1ncccc1-c1cnc(NCc2c(F)ccc3c2CCO3)n2cnnc12]

In addition, the invention provides the use of such dosage and dosing regimens for treating PRC2-mediated diseases or disorders.

The invention provides the following aspects listed in the following items:
Item 1: A method for treating a PRC2-mediated disease or disorder in a subject in need thereof, comprising administering a therapeutically effective amount of Compound (1), or a pharmaceutically acceptable salt thereof,

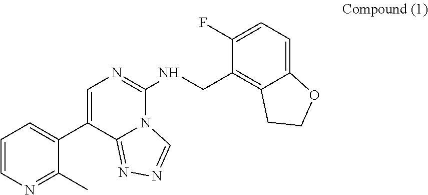 [SMILES: Cc1ncccc1-c1cnc(NCc2c(F)ccc3c2CCO3)n2cnnc12]

wherein Compound (1), or a pharmaceutically acceptable salt thereof, is administered at least once a day at a dose of about 10 mg to about 800 mg, or Compound (1), or a pharmaceutically acceptable salt thereof, is administered twice a day at a dose of about 60 mg to about 300 mg.
Item 2: Compound (1), or a pharmaceutically acceptable salt thereof, for use in the treatment of a PRC2-mediated disease or disorder,

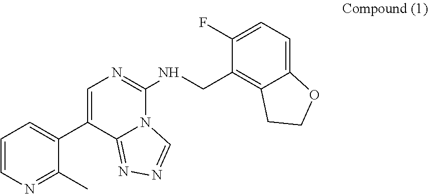 [SMILES: Cc1ncccc1-c1cnc(NCc2c(F)ccc3c2CCO3)n2cnnc12]

wherein Compound (1), or a pharmaceutically acceptable salt thereof, is used once a day at a dose of about 10 mg to about 800 mg, or Compound (1), or a pharmaceutically acceptable salt thereof, is used twice a day at a dose of about 60 mg to about 300 mg.
Item 3: Use of Compound (1), or a pharmaceutically acceptable salt thereof, in the treatment of a PRC2-mediated disease or disorder,

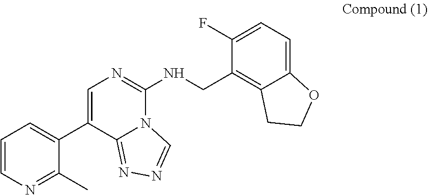 [SMILES: Cc1ncccc1-c1cnc(NCc2c(F)ccc3c2CCO3)n2cnnc12]

wherein Compound (1), or a pharmaceutically acceptable salt thereof, is used once a day at a dose of about 10 mg to about 800 mg, or Compound (1), or a pharmaceutically acceptable salt thereof, is used twice a day at a dose of about 60 mg to about 300 mg.
Item 4: Use of a compound (1), or a pharmaceutically acceptable salt thereof, in the manufacture of a medicament for the treatment of a PRC2-mediated disease or disorder,

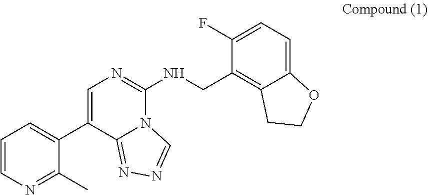 [SMILES: Cc1ncccc1-c1cnc(NCc2c(F)ccc3c2CCO3)n2cnnc12]

wherein Compound (1), or a pharmaceutically acceptable salt thereof, is present in an amount of 2.5 mg to about 100 mg.
Item 5: A medicament comprising Compound (1), or a pharmaceutically acceptable salt thereof, wherein Compound (1), or a pharmaceutically acceptable salt thereof, is present in an amount of about 2.5 mg to about 100 mg.
Item 6: A therapeutic regimen for treating a PRC2-mediated disease or disorder, comprising administering Compound (1), or pharmaceutically acceptable salt thereof, once a day at a dose of about 10 mg to about 800 mg, or Compound (1), or a pharmaceutically acceptable salt thereof, is administered twice a day at a dose of about 60 mg to about 300 mg.
Item 7: A method for treating a PRC2-mediated disease or disorder in a subject in need thereof, comprising administering a therapeutically effective amount of Compound (1), or a pharmaceutically acceptable salt thereof,

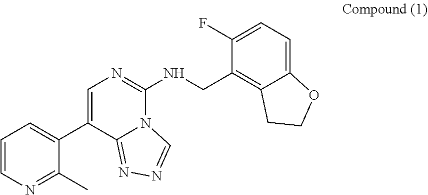 [SMILES: Cc1ncccc1-c1cnc(NCc2c(F)ccc3c2CCO3)n2cnnc12]

wherein Compound (1), or a pharmaceutically acceptable salt thereof, is administered once a day in an amount sufficient to provide Compound (1) at a dose of about 10 mg to about 800 mg, or Compound (1), or a pharmaceutically acceptable salt thereof, is administered twice a day in an amount sufficient to provide Compound (1) at a dose of about 60 mg to about 300 mg.
Item 8: Compound (1), or a pharmaceutically acceptable salt thereof, for use in the treatment of a PRC2-mediated disease or disorder,

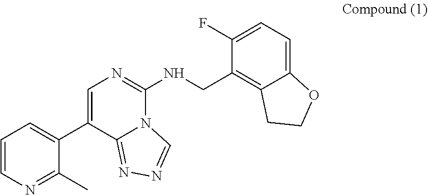 [SMILES: Cc1ncccc1-c1cnc(NCc2c(F)ccc3c2CCO3)n2cnnc12]

wherein Compound (1), or a pharmaceutically acceptable salt thereof, is used once a day in an amount sufficient to provide Compound (1) at a dose of about 10 mg to about 800 mg, or Compound (1), or a pharmaceutically acceptable salt thereof, is used twice a day in an amount sufficient to provide Compound (1) at a dose of about 60 mg to about 300 mg.
Item 9: Use of Compound (1), or a pharmaceutically acceptable salt thereof, in the treatment of a PRC2-mediated disease or disorder,

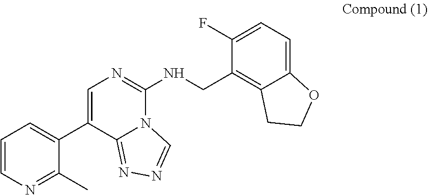 [SMILES: Cc1ncccc1-c1cnc(NCc2c(F)ccc3c2CCO3)n2cnnc12]

wherein Compound (1), or a pharmaceutically acceptable salt thereof, is used once a day in an amount sufficient to provide Compound (1) at a dose of about 10 mg to about 800 mg, or Compound (1), or a pharmaceutically acceptable salt thereof, is used twice a day in an amount sufficient to provide Compound (1) at a dose of about 60 mg to about 300 mg.
Item 10: Use of a compound (1), or a pharmaceutically acceptable salt thereof, in the manufacture of a medicament for the treatment of a PRC2-mediated disease or disorder,

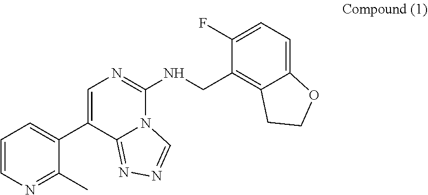 [SMILES: Cc1ncccc1-c1cnc(NCc2c(F)ccc3c2CCO3)n2cnnc12]

wherein Compound (1), or a pharmaceutically acceptable salt thereof, is present in an amount of 2.5 mg to about 100 mg.
Item 11: A medicament comprising Compound (1), or a pharmaceutically acceptable salt thereof, wherein Compound (1), or a pharmaceutically acceptable salt thereof, is present in an amount of about 2.5 mg to about 100 mg.
Item 12: A therapeutic regimen for treating a PRC2-mediated disease or disorder, comprising administering Compound (1), or pharmaceutically acceptable salt thereof, once a day in an amount sufficient to provide Compound (1) at a dose of about 10 mg to about 800 mg, or Compound (1), or a pharmaceutically acceptable salt thereof, is administered twice a day in an amount sufficient to provide Compound (1) at a dose of about 60 mg to about 300 mg.

### DETAILED DESCRIPTION OF THE INVENTION

### Definitions

The phrase “amount sufficient to provide Compound (1)”, as used herein, refers to a the mass of N-((5-fluoro-2,3-dihydrobenzofuran-4-yl)methyl)-8-(2-methylpyridin-3-yl)-[1,2,4]triazolo[4,3-c]pyrimidin-5-amine (Compound (1)), or a pharmaceutically acceptable salt thereof, administered to ensure that the desired dosage of free form N-((5-fluoro-2,3-dihydrobenzofuran-4-yl)methyl)-8-(2-methylpyridin-3-yl)-[1,2,4]triazolo[4,3-c]pyrimidin-5-amine is administered.

The terms “composition” or “pharmaceutical composition,” as used herein, refers to a mixture of Compound (1), or pharmaceutically acceptable salt thereof, and one or more one or more pharmaceutically acceptable carriers, in a form suitable for oral or parenteral administration.

The terms “carrier(s)” or “pharmaceutically acceptable carrier(s)”, as used herein, includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated.

The phrase “pharmaceutically acceptable”, as used herein, means a nontoxic material that does not interfere with the effectiveness of the biological activity of the active ingredient(s).

The term “patient” or “subject” as used herein, encompasses mammals and non-mammals. Examples of mammals include, but are not limited to, humans, chimpanzees, apes, monkeys, cattle, horses, sheep, goats, swine; rabbits, dogs, cats, rats, mice, guinea pigs, and the like. Examples of non-mammals include, but are not limited to, birds, fish and the like. Frequently the patient or subject is a human.

The term “a patient in need”, refers to a patient, which would benefit biologically, medically or in quality of life from treatment with Compound (1).

The term “therapeutically effective amount”, as used herein, refers to an amount of the Compound (1), or a pharmaceutically acceptable salt thereof, which is sufficient to achieve the stated effect. Accordingly, a therapeutically effective amount of Compound (1), or a pharmaceutically acceptable salt thereof, used for the treatment of a PRC2-mediated disease or disorder will be an amount sufficient for the treatment of a PRC2-mediated disease or disorder.

The term “treat”, “treating”, “treatment” or “therapy” as used herein comprises a treatment relieving, reducing or alleviating at least one symptom in a patient, or effecting a delay of progression of a disease in a patient. For example, treatment can be the diminishment of one or several symptoms of a disorder or complete eradication of a disorder, such as cancer. Within the meaning of the present invention, the term “treat” also denotes to arrest, delay the onset (i.e., the period prior to clinical manifestation of a disease) and/or reduce the risk of developing or worsening a disease.

The term “therapeutic regimen”, as used herein, refers to the pattern of dosing used during the treatment of the disease or disorder and is also referred to as “dosing regimen” or “dosing schedule”.

The term “about”, “approximately”, or “approximate”, when used in connection with a numerical value, means that a collection or range of values is included. For example, “about X” includes a range of values that are ±20%, ±10%, ±5%, ±2%, ±1%, ±0.5%, ±0.2%, or ±0.1% of X, where X is a numerical value. In one embodiment, the term “about” refers to a range of values which are 10% more or less than the specified value. In another embodiment, the term “about” refers to a range of values which are 5% more or less than the specified value. In another embodiment, the term “about” refers to a range of values which are 1% more or less than the specified value.

Recitation of ranges of values are merely intended to serve as a shorthand method of referring individually to each separate value falling within the range, unless otherwise indicated herein, and each separate value is incorporated into the specification as if it were individually recited herein. A range used herein, unless otherwise specified, includes the two limits of the range. For example, the terms “between X and Y” and “range from X to Y, are inclusive of X and Y and the integers there between. On the other hand, when a series of individual values are referred to in the disclosure, any range including any of the two individual values as the two end points is also conceived in this disclosure. For example, the expression “a dose of about 10 mg, about 20 mg, about 40 mg, about 80 mg, about 120 mg, about 160 mg, about 240 mg, about 300 mg, about 500 mg or about 800 mg”, also means “a dose ranging from 10 to 800 mg”.

The compound names provided herein were obtained using ChemBioDraw Ultra 14.0 (CambridgeSoft®).

As used herein, the term “a,” “an,” “the” and similar terms used in the context of the present invention (especially in the context of the claims) are to be construed to cover both the singular and plural unless otherwise indicated herein or clearly contradicted by the context.

### Description of Embodiments of the Invention

The invention provides dosage and dosing regimens for N-((5-fluoro-2,3-dihydrobenzofuran-4-yl)methyl)-8-(2-methylpyridin-3-yl)-[1,2,4]triazolo[4,3-c]pyrimidin-5-amine (Compound 1), or a pharmaceutically acceptable salt thereof, for treating PRC2-mediated diseases or disorders. N-((5-fluoro-2,3-dihydrobenzofuran-4-yl)methyl)-8-(2-methylpyridin-3-yl)-[1,2,4]triazolo[4,3-c]pyrimidin-5-amine (Compound 1) has the following structure:

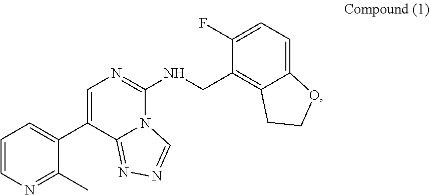 [SMILES: Cc1ncccc1-c1cnc(NCc2c(F)ccc3c2CCO3)n2cnnc12]

and the preferred pharmaceutically acceptable salt is the hydrochloride salt of N-((5-fluoro-2,3-dihydrobenzofuran-4-yl)methyl)-8-(2-methylpyridin-3-yl)-[1,2,4]triazolo[4,3-c] pyrimidin-5-amine (Compound 2), which has the following structure:

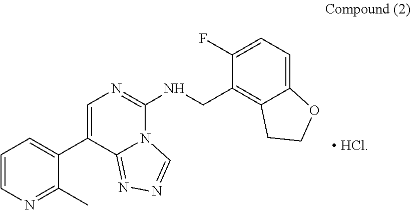

### Salt Forms

N-((5-fluoro-2,3-dihydrobenzofuran-4-yl)methyl)-8-(2-methylpyridin-3-yl)-[1,2,4]triazolo[4,3-c]pyrimidin-5-amine used in the dosages and dosing regimens provided herein can be a salt, in particular a pharmaceutically acceptable salt. Specifically, N-((5-fluoro-2,3-dihydrobenzofuran-4-yl)methyl)-8-(2-methylpyridin-3-yl)-[1,2,4]triazolo[4,3-c]pyrimidin-5-amine used in the dosages and dosing regimens provided herein can be an acid addition salt, in particular a pharmaceutically acceptable acid addition salt.

Pharmaceutically acceptable acid addition salts can be formed with inorganic acids and organic acids, such as hydrochloric acid, hydrobromic acid, sulfuric acid, nitric acid, phosphoric acid, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid and sulfosalicylic acid.

Although any acid addition salt of N-((5-fluoro-2,3-dihydrobenzofuran-4-yl)methyl)-8-(2-methylpyridin-3-yl)-[1,2,4]triazolo[4,3-c]pyrimidin-5-amine can be used in the dosages and dosing regimens provided herein, the preferred pharmaceutically acceptable acid addition salt of N-((5-fluoro-2,3-dihydrobenzofuran-4-yl)methyl)-8-(2-methylpyridin-3-yl)-[1,2,4]triazolo[4,3-c]pyrimidin-5-amine is the hydrochloride salt (Compound 2):

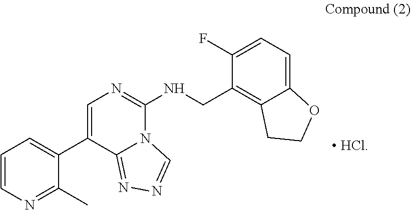

Thus, the compound of Formula (1) of any one of the Embodiments provided herein is the N-((5-fluoro-2,3-dihydrobenzofuran-4-yl)methyl)-8-(2-methylpyridin-3-yl)-[1,2,4]triazolo[4,3-c]pyrimidin-5-amine hydrochloride salt.

Unless otherwise specified herein, the dosage values for the uses and methods of treatment using Compound (1), or a pharmaceutically acceptable salt thereof, described herein refer to the amount of Compound (1) or the amount of the pharmaceutically acceptable salt in the dosage form administered.

However, in certain embodiments the dosage of Compound (1) is based on the amount of free form Compound (1) present in the dosage form rather than the amount of the pharmaceutically acceptable salt, then the dosage values disclosed herein with respect to N-((5-fluoro-2,3-dihydrobenzofuran-4-yl)methyl)-8-(2-methylpyridin-3-yl)-[1,2,4]triazolo[4,3-c]pyrimidin-5-amine, or a pharmaceutically acceptable salt thereof, refer to the mass for the free form (i.e. neutral form) of N-((5-fluoro-2,3-dihydrobenzofuran-4-yl)methyl)-8-(2-methylpyridin-3-yl)-[1,2,4]triazolo[4,3-c]pyrimidin-5-amine administered and not to the mass of the salt form administered. Consequently, the mass of N-((5-fluoro-2,3-dihydrobenzofuran-4-yl)methyl)-8-(2-methylpyridin-3-yl)-[1,2,4]triazolo[4,3-c]pyrimidin-5-amine in a salt form used in the dosing regimens, methods, compositions, and uses disclosed herein for the administration of a specified dose of N-((5-fluoro-2,3-dihydrobenzofuran-4-yl)methyl)-8-(2-methylpyridin-3-yl)-[1,2,4]triazolo[4,3-c]pyrimidin-5-amine is different from the mass of free form N-((5-fluoro-2,3-dihydrobenzofuran-4-yl)methyl)-8-(2-methylpyridin-3-yl)-[1,2,4]triazolo[4,3-c]pyrimidin-5-amine administered. The mass needed for N-((5-fluoro-2,3-dihydrobenzofuran-4-yl)methyl)-8-(2-methylpyridin-3-yl)-[1,2,4]triazolo[4,3-c]pyrimidin-5-amine in a salt form may be calculated based on the ratio of the molecular weights of the salt form and the free form of N-((5-fluoro-2,3-dihydrobenzofuran-4-yl)methyl)-8-(2-methylpyridin-3-yl)-[1,2,4]triazolo[4,3-c]pyrimidin-5-amine. For such embodiments the dosage values is described as the dosage obtained from the administration of Compound (1), or a pharmaceutically acceptable salt thereof, in an amount sufficient to provide Compound (1) at the specified dosage. By way of example, for the administration of a 10 mg dosage of N-((5-fluoro-2,3-dihydrobenzofuran-4-yl)methyl)-8-(2-methylpyridin-3-yl)-[1,2,4]triazolo[4,3-c]pyrimidin-5-amine, wherein the pharmaceutical unit dosage form contains N-((5-fluoro-2,3-dihydrobenzofuran-4-yl)methyl)-8-(2-methylpyridin-3-yl)-[1,2,4]triazolo[4,3-c]pyrimidin-5-amine in free form, the dosage form would contain 10 mg of free form N-((5-fluoro-2,3-dihydrobenzofuran-4-yl)methyl)-8-(2-methylpyridin-3-yl)-[1,2,4]triazolo[4,3-c]pyrimidin-5-amine. However, for the administration of a 10 mg dosage of N-((5-fluoro-2,3-dihydrobenzofuran-4-yl)methyl)-8-(2-methylpyridin-3-yl)-[1,2,4]triazolo[4,3-c]pyrimidin-5-amine, wherein the pharmaceutical unit dosage form contains N-((5-fluoro-2,3-dihydrobenzofuran-4-yl)methyl)-8-(2-methylpyridin-3-yl)-[1,2,4]triazolo[4,3-c]pyrimidin-5-amine hydrochloride salt, then in order to provide the expected dose of 10 mg of N-((5-fluoro-2,3-dihydrobenzofuran-4-yl)methyl)-8-(2-methylpyridin-3-yl)-[1,2,4]triazolo[4,3-c]pyrimidin-5-amine the dosage form would contain 10.97 mg of the hydrochloride salt form. This is further illustrated for other dosage values in Table 1 below:

TABLE 1

| | Mass of free form | Mass of hydrochloride |
|---|---|---|
| Dosage of | of Compound (1) | salt form of Compound (1) |
| Compound (1) | in dosage form | in dosage form |
| (mg) | (mg) | (mg) |
| 2.5 | 2.5 | 2.74 |
| 10 | 10 | 10.97 |
| 50 | 50 | 54.84 |
| 100 | 100 | 109.7 |

Incorporation of certain isotopes, particularly deuterium (i.e., ²H or D) may afford certain therapeutic advantages resulting from greater metabolic stability, for example increased in vivo half-life or reduced dosage requirements or an improvement in therapeutic index or tolerability. It is understood that deuterium in this context is regarded as a substituent of a compound of the present invention. The concentration of deuterium, may be defined by the isotopic enrichment factor. The term “isotopic enrichment factor” as used herein means the ratio between the isotopic abundance and the natural abundance of a specified isotope. If a substituent in a compound of this invention is denoted as being deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). It should be understood that the term “isotopic enrichment factor” can be applied to any isotope in the same manner as described for deuterium.

Other examples of isotopes that can be incorporated into compounds of the invention include isotopes of hydrogen, carbon, nitrogen, oxygen, phosphorous, fluorine, and chlorine, such as ³H, ¹¹C, ¹³C, ¹⁴C, ¹⁵N, ¹⁸F ³¹P, ³²P, ³⁵S, ³⁶Cl, ¹²³I, ¹²⁴I, ¹²⁵I respectively. Accordingly it should be understood that the invention includes compounds that incorporate one or more of any of the aforementioned isotopes, including for example, radioactive isotopes, such as ³H and ¹⁴C, or those into which non-radioactive isotopes, such as ²H and ¹³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies. Isotopically-labeled compounds of the present invention can generally be prepared by conventional techniques known to those skilled in the art or by processes analogous to those described in the accompanying Examples and Preparations using an appropriate isotopically-labeled reagents in place of the non-labeled reagent previously employed.

Dosing regimens (i.e dosing schedules) for the administration of Compound (1), or a pharmaceutically acceptable salt thereof, can be the daily administration of Compound (1), or a pharmaceutically acceptable salt thereof, wherein Compound (1), or a pharmaceutically acceptable salt thereof, is administered at least once a day, or Compound (1), or a pharmaceutically acceptable salt thereof, is administered twice a day.

In certain embodiments such dosing schedules are continuous dosing schedules, wherein Compound (1), or a pharmaceutically acceptable salt thereof, is administered daily during a cycle period.

A cycle period is the number and timing, or recommended repetitions, of therapy and are usually expressed as the number of days. Examples of a cycle period include about every 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days or 31 days. In a preferred embodiment, the cycle period is up to 8 days.

In certain embodiments such dosing schedules are continuous dosing schedules, wherein compound (1), or a pharmaceutically acceptable salt thereof, is administered daily for up to about 1, 2, 3, 4, 5, 6, 7, 8, 9 or 10 cycle periods.

A dosing schedule can include a dose delay (drug holiday), wherein Compound (1), or a pharmaceutically acceptable salt thereof, is not administered during one or more cycle periods. Such dose delays can be for up to about 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days or 31 days, or longer.

In certain embodiments dosing schedules are cyclical dosing schedules comprising initial administration of Compound (1), or a pharmaceutically acceptable salt thereof, for one or more cycle periods followed by a dose delay for one or more subsequent cycle periods, and then followed by administration of Compound (1), or a pharmaceutically acceptable salt thereof, for one or more additional cycle periods. Such dosing schedules can be used to help mitigate treatment related events that would preclude a continuous dosing schedule for the same period of time as that obtainable using a cyclic dosing schedule.

An embodiment of a cyclical dosing schedule is the administration of Compound (1), or a pharmaceutically acceptable salt thereof, for one or more cycle periods of up to 28 days.

In another embodiment of a cyclical dosing schedule is the administration of Compound (1), or a pharmaceutically acceptable salt thereof, for 8 consecutive days for one or more cycle periods, followed by a dose delay of 8 days for one or more cycle periods, and then further administration of Compound (1), or a pharmaceutically acceptable salt thereof, for 8 consecutive days for one or more cycle periods.

In another embodiment of a cyclical dosing schedule is the administration of Compound (1), or a pharmaceutically acceptable salt thereof, for 8 consecutive days for a cycle period followed by a dose delay of 8 days for a cycle period and then further administration of Compound (1), or a pharmaceutically acceptable salt thereof, for 8 consecutive days for a cycle period.

In preferred embodiment of a cyclical dosing schedule is the administration of Compound (1), or a pharmaceutically acceptable salt thereof, for 8 consecutive days for an eight day cycle period followed by a dose delay of 8 days for an eight day cycle period and then further administration of Compound (1), or a pharmaceutically acceptable salt thereof, for 8 consecutive days for an eight day cycle period.

Another embodiment of cyclical dosing schedule is the administration of Compound (1), or a pharmaceutically acceptable salt thereof, for 14 consecutive days followed by a dose delay of 14 days and then further administration of Compound (1), or a pharmaceutically acceptable salt thereof, for 10 consecutive days. This cyclic dosing schedule is also referred to as 14 days on; 14 days off; 10 days on.

Certain aspects and examples of dosage and dosing regimens (therapeutic regimen) for Compound (1), or a pharmaceutical acceptable salt thereof, are provided in the following listing of enumerated embodiments. It is recognized that features specified in each embodiment may be combined with other specified features to provide further embodiments of such dosage and dosage regimens used to treat PRC2-mediated diseases or disorders.
Embodiment 1. A therapeutic regimen for treating a PRC2-mediated disease or disorder, comprising administering Compound (1), or a pharmaceutically acceptable salt thereof, daily.
Embodiment 2. The therapeutic regimen of Embodiment 1, wherein Compound (1), or a pharmaceutically acceptable salt thereof, is administered at least once a day or twice a day.
Embodiment 3. The therapeutic regimen of Embodiment 1 or Embodiment 2, wherein the therapeutic regimen is a continuous dosing schedule.
Embodiment 4. The therapeutic regimen of Embodiment 3, wherein the continuous dosing schedule comprises administering Compound (1), or a pharmaceutically acceptable salt thereof, for 7 consecutive days, 8 consecutive days, 9 consecutive days, 10 consecutive days, 11 consecutive days, 12 consecutive days, 13 consecutive days, 14 consecutive days, 15 consecutive days, 16 consecutive days, 17 consecutive days, 18 consecutive days, 19 consecutive days, 20 consecutive days, 21 consecutive days, 22 consecutive days, 23 consecutive days, 24 consecutive days, 25 consecutive days, 26 consecutive days, 27 consecutive days, 28 consecutive days, 29 consecutive days, 30 consecutive days or 31 consecutive days.
Embodiment 5. The therapeutic regimen of Embodiment 3 or Embodiment 4, wherein the continuous dosing schedule comprises administering Compound (1), or a pharmaceutically acceptable salt thereof, for 8 consecutive days.
Embodiment 6. The therapeutic regimen of Embodiment 1 or Embodiment 2, wherein Compound (1), or a pharmaceutically acceptable salt thereof, is administered for 7 consecutive days, 8 consecutive days, 9 consecutive days, 10 consecutive days, 11 consecutive days, 12 consecutive days, 13 consecutive days, 14 consecutive days, 15 consecutive days, 16 consecutive days, 17 consecutive days, 18 consecutive days, 19 consecutive days, 20 consecutive days, 21 consecutive days, 22 consecutive days, 23 consecutive days, 24 consecutive days, 25 consecutive days, 26 consecutive days, 27 consecutive days, 28 consecutive days, 29 consecutive days, 30 consecutive days or 31 consecutive days.
Embodiment 7. The therapeutic regimen of any one of Embodiments 1, 2, 3 or 6, wherein Compound (1), or a pharmaceutically acceptable salt thereof, is administered for 8 consecutive days.
Embodiment 8. The therapeutic regimen of any one of Embodiments 1 to 7, wherein further comprises administering Compound (1), or a pharmaceutically acceptable salt thereof, for one or more cycle periods.
Embodiment 9. The therapeutic regimen of Embodiment 8, wherein a cycle period is 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days or 31 days.
Embodiment 10. The therapeutic regimen of Embodiment 8 or Embodiment 9, wherein a cycle period is 8 days.
Embodiment 11. The therapeutic regimen of any one of Embodiments 8 to 10, wherein Compound (1), or a pharmaceutically acceptable salt thereof, is administered for two cycle periods and each cycle period is 8 days.
Embodiment 12. The therapeutic regimen of any one of Embodiments 1 to 11, wherein the therapeutic regimen further comprises a dose delay.
Embodiment 13. The therapeutic regimen of Embodiment 12, wherein the dose delay is for 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days or 31 days, or longer.
Embodiment 14. The therapeutic regimen of any one of Embodiments 1 to 13, wherein Compound (1), or a pharmaceutically acceptable salt thereof, is administered for two cycle periods with a dose delay.
Embodiment 15. The therapeutic regimen of Embodiment 14, wherein each cycle period is 8 days and the dose delay is in between the first and second cycle period.
Embodiment 16. The therapeutic regimen of Embodiment 15, wherein Compound (1), or a pharmaceutically acceptable salt thereof, is administered for 8 consecutive days of the first 8 day cycle period, followed by a dose delay of 8 days, then followed by administration of Compound (1), or a pharmaceutically acceptable salt thereof, for 8 consecutive days of the second eight day cycle period.
Embodiment 17. The therapeutic regimen of Embodiment 1 or Embodiment 2, wherein the therapeutic regimen is a cyclical dosing schedule comprising administering Compound (1), or a pharmaceutically acceptable salt thereof, for one or more cycle periods followed by a dose delay of one or more cycle periods, then followed by administering Compound (1), or a pharmaceutically acceptable salt thereof, for an additional one or more cycle periods.
Embodiment 18. The therapeutic regimen of Embodiment 17, wherein a cycle period is 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days or 31 days.
Embodiment 19. The therapeutic regimen of Embodiment 17 or Embodiment 18, wherein the dose delay is for 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days or 31 days, or longer.
Embodiment 20. The therapeutic regimen of any one of Embodiments 17 to 19, wherein Compound (1), or a pharmaceutically acceptable salt thereof, is administered for a cycle period, followed by a dose delay and then followed by administering Compound (1), or a pharmaceutically acceptable salt thereof, for an additional cycle period.
Embodiment 21. The therapeutic regimen of Embodiment 20, wherein each cycle period is 8 days and the dose delay is eight days.
Embodiment 22. The therapeutic regimen of any one of Embodiments 17 to 21, wherein Compound (1), or a pharmaceutically acceptable salt thereof, is administered for 8 consecutive days of the first 8 day cycle period, followed by a dose delay of 8 days, then followed by administration of Compound (1), or a pharmaceutically acceptable salt thereof, for 8 consecutive days of the second eight day cycle period.
Embodiment 23. The therapeutic regimen of any one of Embodiments 1 to 22, wherein Compound (1), or a pharmaceutically acceptable salt thereof, is administered once a day at a dose of about 10 mg to about 800 mg, or Compound (1), or a pharmaceutically acceptable salt thereof, is administered twice a day at a dose of about 60 mg to about 300 mg.
Embodiment 24. The therapeutic regimen of any one of Embodiments 1 to 23, wherein Compound (1), or a pharmaceutically acceptable salt thereof, is administered once a day at a dose of about 10 mg to about 800 mg.
Embodiment 25. The therapeutic regimen of any one of Embodiments 1 to 24, wherein Compound (1), or a pharmaceutically acceptable salt thereof, is administered once a day at a dose of about 10 mg, about 20 mg, about 40 mg, about 80 mg, about 120 mg, about 160 mg, about 240 mg, about 300 mg, about 500 mg or about 800 mg.
Embodiment 26. The therapeutic regimen of any one of Embodiments 1 to 24, wherein Compound (1), or a pharmaceutically acceptable salt thereof, is administered once a day at a dose of about 10 mg.
Embodiment 27. The therapeutic regimen of any one of Embodiments 1 to 24, wherein Compound (1), or a pharmaceutically acceptable salt thereof, is administered once a day at a dose of about 20 mg.
Embodiment 28. The therapeutic regimen of any one of Embodiments 1 to 24, wherein Compound (1), or a pharmaceutically acceptable salt thereof, is administered once a day at a dose of about 40 mg.
Embodiment 29. The therapeutic regimen of any one of Embodiments 1 to 24, wherein Compound (1), or a pharmaceutically acceptable salt thereof, is administered once a day at a dose of about 80 mg.
Embodiment 30. The therapeutic regimen of any one of Embodiments 1 to 24, wherein Compound (1), or a pharmaceutically acceptable salt thereof, is administered once a day at a dose of about 120 mg.
Embodiment 31. The therapeutic regimen of any one of Embodiments 1 to 24, wherein Compound (1), or a pharmaceutically acceptable salt thereof, is administered once a day at a dose of about 160 mg.
Embodiment 32. The therapeutic regimen of any one of Embodiments 1 to 24, wherein Compound (1), or a pharmaceutically acceptable salt thereof, is administered once a day at a dose of about 240 mg.
Embodiment 33. The therapeutic regimen of any one of Embodiments 1 to 24, wherein Compound (1), or a pharmaceutically acceptable salt thereof, is administered once a day at a dose of about 300 mg.
Embodiment 34. The therapeutic regimen of any one of Embodiments 1 to 24, wherein Compound (1), or a pharmaceutically acceptable salt thereof, is administered once a day at a dose of about 500 mg.
Embodiment 35. The therapeutic regimen of any one of Embodiments 1 to 24, wherein Compound (1), or a pharmaceutically acceptable salt thereof, is administered once a day at a dose of about 800 mg.
Embodiment 36. The therapeutic regimen of any one of Embodiments 1 to 24, wherein Compound (1), or a pharmaceutically acceptable salt thereof, is administered once a day at a dose of about 10 mg for eight consecutive days.
Embodiment 37. The therapeutic regimen of any one of Embodiments 1 to 24, wherein Compound (1), or a pharmaceutically acceptable salt thereof, is administered once a day at a dose of about 20 mg for eight consecutive days.
Embodiment 38. The therapeutic regimen of any one of Embodiments 1 to 24, wherein Compound (1), or a pharmaceutically acceptable salt thereof, is administered once a day at a dose of about 40 mg for eight consecutive days.
Embodiment 39. The therapeutic regimen of any one of Embodiments 1 to 24, wherein Compound (1), or a pharmaceutically acceptable salt thereof, is administered once a day at a dose of about 80 mg for eight consecutive days.
Embodiment 40. The therapeutic regimen of any one of Embodiments 1 to 24, wherein Compound (1), or a pharmaceutically acceptable salt thereof, is administered once a day at a dose of about 120 mg for eight consecutive days.
Embodiment 41. The therapeutic regimen of any one of Embodiments 1 to 24, wherein Compound (1), or a pharmaceutically acceptable salt thereof, is administered once a day at a dose of about 160 mg for eight consecutive days.
Embodiment 42. The therapeutic regimen of any one of Embodiments 1 to 24, wherein Compound (1), or a pharmaceutically acceptable salt thereof, is administered once a day at a dose of about 240 mg for eight consecutive days.
Embodiment 43. The therapeutic regimen of any one of Embodiments 1 to 24, wherein Compound (1), or a pharmaceutically acceptable salt thereof, is administered once a day at a dose of about 300 mg for eight consecutive days.
Embodiment 44. The therapeutic regimen of any one of Embodiments 1 to 24, wherein Compound (1), or a pharmaceutically acceptable salt thereof, is administered once a day at a dose of about 500 mg for eight consecutive days.
Embodiment 45. The therapeutic regimen of any one of Embodiments 1 to 24, wherein Compound (1), or a pharmaceutically acceptable salt thereof, is administered once a day at a dose of about 800 mg for eight consecutive days.
Embodiment 46. The therapeutic regimen of any one of Embodiments 1 to 22, wherein Compound (1), or a pharmaceutically acceptable salt thereof, is administered twice a day at a dose of about at a dose of about 60 mg to about 300 mg.
Embodiment 47. The therapeutic regimen of any one of Embodiments 1 to 22 or Embodiment 40, wherein Compound (1), or a pharmaceutically acceptable salt thereof, is administered twice a day at a dose of about 60 mg, about 80 mg, about 120 mg, about 150 mg, or about 300 mg.
Embodiment 48. The therapeutic regimen of any one of Embodiments 1 to 22 or Embodiments 40 to 41, wherein Compound (1), or a pharmaceutically acceptable salt thereof, is administered twice a day at a dose of about 60 mg.
Embodiment 49. The therapeutic regimen of any one of Embodiments 1 to 22 or Embodiments 40 to 41, wherein Compound (1), or a pharmaceutically acceptable salt thereof, is administered twice a day at a dose of about 80 mg.
Embodiment 50. The therapeutic regimen of any one of Embodiments 1 to 22 or Embodiments 40 to 41, wherein compound (1), or a pharmaceutically acceptable salt thereof, is administered twice a day at a dose of about 120 mg.
Embodiment 51. The therapeutic regimen of any one of Embodiments 1 to 22 or Embodiments 40 to 41, wherein Compound (1), or a pharmaceutically acceptable salt thereof, is administered twice a day at a dose of about 150 mg.
Embodiment 52. The therapeutic regimen of any one of Embodiments 1 to 22 or Embodiments 40 to 41, wherein Compound (1), or a pharmaceutically acceptable salt thereof, is administered twice a day at a dose of about 300 mg.
Embodiment 53. The therapeutic regimen of any one of Embodiments 1 to 22, wherein Compound (1), or a pharmaceutically acceptable salt thereof, is administered twice a day at a dose of about at a dose of about 60 mg to about 300 mg for eight consecutive days.
Embodiment 54. The therapeutic regimen of any one of Embodiments 1 to 22 or Embodiment 40, wherein Compound (1), or a pharmaceutically acceptable salt thereof, is administered twice a day at a dose of about 60 mg, about 80 mg, about 120 mg, about 150 mg, or about 300 mg for eight consecutive days.
Embodiment 55. The therapeutic regimen of any one of Embodiments 1 to 22 or Embodiments 40 to 41, wherein Compound (1), or a pharmaceutically acceptable salt thereof, is administered twice a day at a dose of about 60 mg for eight consecutive days.
Embodiment 56. The therapeutic regimen of any one of Embodiments 1 to 22 or Embodiments 40 to 41, wherein Compound (1), or a pharmaceutically acceptable salt thereof, is administered twice a day at a dose of about 80 mg for eight consecutive days.
Embodiment 57. The therapeutic regimen of any one of Embodiments 1 to 22 or Embodiments 40 to 41, wherein compound (1), or a pharmaceutically acceptable salt thereof, is administered twice a day at a dose of about 120 mg for eight consecutive days.
Embodiment 58. The therapeutic regimen of any one of Embodiments 1 to 22 or Embodiments 40 to 41, wherein Compound (1), or a pharmaceutically acceptable salt thereof, is administered twice a day at a dose of about 150 mg for eight consecutive days.
Embodiment 59. The therapeutic regimen of any one of Embodiments 1 to 22 or Embodiments 40 to 41, wherein Compound (1), or a pharmaceutically acceptable salt thereof, is administered twice a day at a dose of about 300 mg for eight consecutive days.
Embodiment 60. The therapeutic regimen of any one of Embodiments 1 to 59, wherein Compound (1) is administered as the hydrochloride salt.
Embodiment 61. The therapeutic regimen of any one of Embodiments 1 to 60, wherein Compound (1), or a pharmaceutically acceptable salt thereof, is orally administered.
Embodiment 62. A therapeutic regimen for treating a PRC2-mediated disease or disorder, comprising administering a Compound (1), or a pharmaceutically acceptable salt thereof, once a day in an amount sufficient to provide Compound (1) at a dose of about 10 mg to about 800 mg, or Compound (1), or a pharmaceutically acceptable salt thereof, is administered twice a day in an amount sufficient to provide Compound (1) at a dose of about 60 mg to about 300 mg.
Embodiment 63. The therapeutic regimen of Embodiment 62, wherein Compound (1), or a pharmaceutically acceptable salt thereof, is administered once a day in an amount sufficient to provide Compound (1) at a dose of about 10 mg to about 800 mg.
Embodiment 64. The therapeutic regimen of Embodiment 62 or Embodiment 63, wherein Compound (1), or a pharmaceutically acceptable salt thereof, is administered once a day in an amount sufficient to provide Compound (1) at a dose of about 10 mg, about 20 mg, about 40 mg, about 80 mg, about 120 mg, about 160 mg, about 240 mg, about 300 mg, about 500 mg or about 800 mg.
Embodiment 65. The therapeutic regimen of any one of Embodiments 62 to 64, wherein Compound (1), or a pharmaceutically acceptable salt thereof, is administered once a day in an amount sufficient to provide Compound (1) at a dose of about 10 mg.
Embodiment 66. The therapeutic regimen of any one of Embodiments 62 to 64, wherein Compound (1), or a pharmaceutically acceptable salt thereof, is administered once a day in an amount sufficient to provide Compound (1) at a dose of about 20 mg.
Embodiment 67. The therapeutic regimen of any one of Embodiments 62 to 64, wherein Compound (1), or a pharmaceutically acceptable salt thereof, is administered once a day in an amount sufficient to provide Compound (1) at a dose of about 40 mg.
Embodiment 68. The therapeutic regimen of any one of Embodiments 62 to 64, wherein Compound (1), or a pharmaceutically acceptable salt thereof, is administered once a day in an amount sufficient to provide Compound (1) at a dose of about 80 mg.
Embodiment 69. The therapeutic regimen of any one of Embodiments 62 to 64, wherein Compound (1), or a pharmaceutically acceptable salt thereof, is administered once a day in an amount sufficient to provide Compound (1) at a dose of about 120 mg.
Embodiment 70. The therapeutic regimen of any one of Embodiments 62 to 64, wherein Compound (1), or a pharmaceutically acceptable salt thereof, is administered once a day in an amount sufficient to provide Compound (1) at a dose of about 160 mg.
Embodiment 71. The therapeutic regimen of any one of Embodiments 62 to 64, wherein Compound (1), or a pharmaceutically acceptable salt thereof, is administered once a day in an amount sufficient to provide Compound (1) at a dose of about 240 mg.
Embodiment 72. The therapeutic regimen of any one of Embodiments 62 to 64, wherein Compound (1), or a pharmaceutically acceptable salt thereof, is administered once a day in an amount sufficient to provide Compound (1) at a dose of about 300 mg.
Embodiment 73. The therapeutic regimen of any one of Embodiments 62 to 64, wherein Compound (1), or a pharmaceutically acceptable salt thereof, is administered once a day in an amount sufficient to provide Compound (1) at a dose of about 500 mg.
Embodiment 74. The therapeutic regimen of any one of Embodiments 62 to 64, wherein Compound (1), or a pharmaceutically acceptable salt thereof, is administered once a day in an amount sufficient to provide Compound (1) at a dose of about 800 mg.
Embodiment 75. The therapeutic regimen of Embodiment 62, wherein Compound (1), or a pharmaceutically acceptable salt thereof, is administered twice a day in an amount sufficient to provide Compound (1) at a dose of about at a dose of about 60 mg to about 300 mg.
Embodiment 76. The therapeutic regimen of Embodiment 62 or Embodiment 75, wherein compound (1), or a pharmaceutically acceptable salt thereof, is administered twice a day in an amount sufficient to provide Compound (1) at a dose of about 60 mg, about 80 mg, about 120 mg, about 150 mg, or about 300 mg.
Embodiment 77. The therapeutic regimen of Embodiment 62 or any one of Embodiments 75 to 76, wherein compound (1), or a pharmaceutically acceptable salt thereof, is administered twice a day in an amount sufficient to provide Compound (1) at a dose of about 60 mg.
Embodiment 78. The therapeutic regimen of Embodiment 62 or any one of Embodiments 75 to 76, wherein compound (1), or a pharmaceutically acceptable salt thereof, is administered twice a day in an amount sufficient to provide Compound (1) at a dose of about 80 mg.
Embodiment 79. The therapeutic regimen of Embodiment 62 or any one of Embodiments 75 to 76, wherein compound (1), or a pharmaceutically acceptable salt thereof, is administered twice a day in an amount sufficient to provide Compound (1) at a dose of about 120 mg.
Embodiment 80. The therapeutic regimen of Embodiment 62 or any one of Embodiments 75 to 76, wherein compound (1), or a pharmaceutically acceptable salt thereof, is administered twice a day in an amount sufficient to provide Compound (1) at a dose of about 150 mg.
Embodiment 81. The therapeutic regimen of Embodiment 62 or any one of Embodiments 75 to 76, wherein compound (1), or a pharmaceutically acceptable salt thereof, is administered twice a day in an amount sufficient to provide Compound (1) at a dose of about 300 mg.
Embodiment 82. The therapeutic regimen of any one of Embodiments 62 to 81, wherein the therapeutic regimen is a continuous dosing regimen.
Embodiment 83. The therapeutic regimen of any one of Embodiments 62 to 82, wherein Compound (10, or a pharmaceutically acceptable salt thereof, is administered for 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days or 31 days.
Embodiment 84. The therapeutic regimen of any one of Embodiments 62 to 83, wherein the therapeutic regimen further comprises a dose delay.
Embodiment 85. The therapeutic regimen of Embodiment 84, wherein the dose delay is for 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days or 31 days, or longer.
Embodiment 86. The therapeutic regimen of any one of Embodiments 62 to 83, wherein the therapeutic regimen is a cyclic dosing schedule and further comprises a dose delay and a subsequent dosing schedule.
Embodiment 87. The therapeutic regimen of Embodiment 86, wherein the dose delay is for 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days or 31 days, or longer.
Embodiment 88. The therapeutic regimen of Embodiment 87, wherein the subsequent dosing schedule is daily dosing for 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days or 31 days.
Embodiment 89. The therapeutic regimen of any one of Embodiments 62 to 88, wherein PRC2-mediated disease or disorder is diffused large B cell lymphoma (DLBCL), follicular lymphoma, leukemia, multiple myeloma, mesothelioma, gastric cancer, malignant rhabdoid tumor, hepatocellular carcinoma, prostate cancer, breast carcinoma, bile duct cancer, gallbladder cancers, bladder carcinoma, neuroblastoma, schwannoma, glioma, glioblastoma, astrocytoma, cervical cancer, colon cancer, melanoma, endometrial cancer, esophageal cancer, head and neck cancer, lung cancer, nasopharyngeal carcinoma, ovarian cancer, pancreatic cancer, renal cell carcinoma, rectal cancer, thyroid cancer, parathyroid tumor, uterine tumor, or soft tissue sarcoma.
Embodiment 90. The therapeutic regimen of any one of Embodiments 62 to 89, wherein the PRC2-mediated disease or disorder is diffused large B cell lymphoma (DLBCL), gastric cancer, prostate cancer, nasopharyngeal carcinoma, ovarian cancer, or soft tissue sarcoma.

### Pharmaceutical Compositions, Medicaments and Dosage Forms

In another aspect, the present invention provides a pharmaceutical composition comprising N-((5-fluoro-2,3-dihydrobenzofuran-4-yl)methyl)-8-(2-methylpyridin-3-yl)-[1,2,4]triazolo[4,3-c]pyrimidin-5-amine, or a pharmaceutically acceptable salt thereof, and one or more pharmaceutically acceptable carriers.

Typically, the pharmaceutical compositions comprising N-((5-fluoro-2,3-dihydrobenzofuran-4-yl)methyl)-8-(2-methylpyridin-3-yl)-[1,2,4]triazolo[4,3-c]pyrimidin-5-amine, or a pharmaceutically acceptable salt thereof, further comprise one or more of:

a) diluents, e.g., lactose, dextrose, sucrose, mannitol, sorbitol, cellulose and/or glycine;

b) lubricants, e.g., silica, talcum, stearic acid, its magnesium or calcium salt and/or polyethyleneglycol;

c) binders, e.g., magnesium aluminum silicate, starch paste, gelatin, tragacanth, methylcellulose, sodium carboxymethylcellulose and/or polyvinylpyrrolidone; if desired

d) disintegrants, e.g., starches, agar, alginic acid or its sodium salt, or effervescent mixtures;

and

e) absorbents, colorants, flavors and sweeteners.

In a certain embodiment the pharmaceutical composition comprising N-((5-fluoro-2,3-dihydrobenzofuran-4-yl)methyl)-8-(2-methylpyridin-3-yl)-[1,2,4]triazolo[4,3-c]pyrimidin-5-amine, or a pharmaceutically acceptable salt thereof, also comprises microcrystalline cellulose, colloidal silicon dioxide and magnesium stearate.

Such pharmaceutical compositions can be formulated for oral administration and can be administered as a pharmaceutical dosage form such as capsules, tablets, pills, granules or powders. In certain embodiments tablets may be either film coated or enteric coated according to methods known in the art.

Accordingly, the present invention also provides pharmaceutical unit dosage forms, such as capsules, tablets, pills, granules or powders, comprising N-((5-fluoro-2,3-dihydrobenzofuran-4-yl)methyl)-8-(2-methylpyridin-3-yl)-[1,2,4]triazolo[4,3-c]pyrimidin-5-amine, or a pharmaceutically acceptable salt thereof, and one or more pharmaceutically acceptable carriers selected from:

a) diluents, e.g., lactose, dextrose, sucrose, mannitol, sorbitol, cellulose and/or glycine;

b) lubricants, e.g., silica, talcum, stearic acid, its magnesium or calcium salt and/or polyethyleneglycol;

c) binders, e.g., magnesium aluminum silicate, starch paste, gelatin, tragacanth, methylcellulose, sodium carboxymethylcellulose and/or polyvinylpyrrolidone; if desired

d) disintegrants, e.g., starches, agar, alginic acid or its sodium salt, or effervescent mixtures;

and

e) absorbents, colorants, flavors and sweeteners.

Certain aspects and examples of such pharmaceutical unit dosage forms are provided in the following listing of enumerated embodiments. It will be recognized that features specified in each embodiment may be combined with other specified features to provide further embodiments of such pharmaceutical unit dosage forms for use to treat PRC2-mediated diseases or disorders.
Embodiment 91. A pharmaceutical unit dosage form for oral administration comprising N-((5-fluoro-2,3-dihydrobenzofuran-4-yl)methyl)-8-(2-methylpyridin-3-yl)-[1,2,4]triazolo[4,3-c]pyrimidin-5-amine, or a pharmaceutically acceptable salt thereof, and one or more pharmaceutically acceptable carriers selected from:

a) diluents, e.g., lactose, dextrose, sucrose, mannitol, sorbitol, cellulose and/or glycine;

b) lubricants, e.g., silica, talcum, stearic acid, its magnesium or calcium salt and/or polyethyleneglycol;

c) binders, e.g., magnesium aluminum silicate, starch paste, gelatin, tragacanth, methylcellulose, sodium carboxymethylcellulose and/or polyvinylpyrrolidone; if desired

d) disintegrants, e.g., starches, agar, alginic acid or its sodium salt, or effervescent mixtures;

and

e) absorbents, colorants, flavors and sweeteners.
Embodiment 92. The pharmaceutical unit dosage form of Embodiment 91, wherein the pharmaceutical unit dosage is a capsule, a tablet, a pill, granules or a powder.
Embodiment 93. The pharmaceutical unit dosage form of Embodiment 91 or Embodiment 92, wherein the pharmaceutical unit dosage is a gelatin capsule.
Embodiment 94. The pharmaceutical dosage form of any one of Embodiments 91 to 93, wherein the pharmaceutical dosage form comprises N-((5-fluoro-2,3-dihydrobenzofuran-4-yl)methyl)-8-(2-methylpyridin-3-yl)-[1,2,4]triazolo[4,3-c]pyrimidin-5-amine, or a pharmaceutically acceptable salt thereof, microcrystalline cellulose, colloidal silicon dioxide and magnesium stearate.
Embodiment 95. The pharmaceutical dosage form of any one of Embodiment 91 to 94, wherein the pharmaceutical unit dosage form is a hard gelatin capsule.

Embodiment 96. The pharmaceutical unit dosage form of any one of Embodiment 91 to 95, wherein the pharmaceutical dosage form is a hard gelatin capsule, and wherein the outer shell of the hard gelatin capsule comprises gelatin, titanium dioxide, and iron oxide.
Embodiment 97. The pharmaceutical unit dosage form of any one of Embodiments 91 to 96, wherein the pharmaceutical unit dosage form comprises about 2.5 mg, about 10 mg, about 50 mg, or about 100 mg of N-((5-fluoro-2,3-dihydrobenzofuran-4-yl)methyl)-8-(2-methylpyridin-3-yl)-[1,2,4]triazolo[4,3-c]pyrimidin-5-amine suitable for oral administration of up to a maximum total dose of 800 mg per day of N-((5-fluoro-2,3-dihydrobenzofuran-4-yl)methyl)-8-(2-methylpyridin-3-yl)-[1,2,4]triazolo[4,3-c]pyrimidin-5-amine.
Embodiment 98. The pharmaceutical unit dosage form of any one of Embodiments 91 to 97, wherein the pharmaceutical unit dosage form is orally administered for the treatment of PRC2-mediated diseases or disorders.
Embodiment 99. The pharmaceutical unit dosage form of any one of Embodiments 91 to 98, wherein PRC2-mediated disease or disorder is diffused large B cell lymphoma (DLBCL), follicular lymphoma, leukemia, multiple myeloma, mesothelioma, gastric cancer, malignant rhabdoid tumor, hepatocellular carcinoma, prostate cancer, breast carcinoma, bile duct cancer, gallbladder cancers, bladder carcinoma, neuroblastoma, schwannoma, glioma, glioblastoma, astrocytoma, cervical cancer, colon cancer, melanoma, endometrial cancer, esophageal cancer, head and neck cancer, lung cancer, nasopharyngeal carcinoma, ovarian cancer, pancreatic cancer, renal cell carcinoma, rectal cancer, thyroid cancer, parathyroid tumor, uterine tumor, or soft tissue sarcoma.
Embodiment 100. The pharmaceutical unit dosage form of any one of Embodiments 91 to 99, wherein the PRC2-mediated disease or disorder is diffused large B cell lymphoma (DLBCL), gastric cancer, prostate cancer, nasopharyngeal carcinoma, ovarian cancer, or soft tissue sarcoma.

In another aspect, the present invention provides the use N-((5-fluoro-2,3-dihydrobenzofuran-4-yl)methyl)-8-(2-methylpyridin-3-yl)-[1,2,4]triazolo[4,3-c]pyrimidin-5-amine, or a pharmaceutically acceptable salt thereof, in the manufacture of a medicament for the treatment of a PRC2-mediated disease or disorder, wherein N-((5-fluoro-2,3-dihydrobenzofuran-4-yl)methyl)-8-(2-methylpyridin-3-yl)-[1,2,4]triazolo[4,3-c]pyrimidin-5-amine, is present in an amount of about 2.5 mg to about 100 mg.

Certain aspects and examples of such uses of N-((5-fluoro-2,3-dihydrobenzofuran-4-yl)methyl)-8-(2-methylpyridin-3-yl)-[1,2,4]triazolo[4,3-c]pyrimidin-5-amine, or a pharmaceutically acceptable salt thereof, in the manufacture of a medicament for the treatment of a PRC2-mediated disease or disorder, are provided in the following listing of enumerated embodiments. It will be recognized that features specified in each embodiment may be combined with other specified features to provide further embodiments of such uses.
Embodiment 101. Use of N-((5-fluoro-2,3-dihydrobenzofuran-4-yl)methyl)-8-(2-methylpyridin-3-yl)-[1,2,4]triazolo[4,3-c]pyrimidin-5-amine, or a pharmaceutically acceptable salt thereof, in the manufacture of a medicament for the treatment of a PRC2-mediated disease or disorder, wherein N-((5-fluoro-2,3-dihydrobenzofuran-4-yl)methyl)-8-(2-methylpyridin-3-yl)-[1,2,4]triazolo[4,3-c]pyrimidin-5-amine is present in an amount of about 2.5 mg to about 100 mg.
Embodiment 102. Use of N-((5-fluoro-2,3-dihydrobenzofuran-4-yl)methyl)-8-(2-methylpyridin-3-yl)-[1,2,4]triazolo[4,3-c]pyrimidin-5-amine, or a pharmaceutically acceptable salt thereof, in the manufacture of a medicament for the treatment of a PRC2-mediated disease or disorder, wherein N-((5-fluoro-2,3-dihydrobenzofuran-4-yl)methyl)-8-(2-methylpyridin-3-yl)-[1,2,4]triazolo[4,3-c]pyrimidin-5-amine is present in an amount of about 2.5 mg, about 10 mg, about 50 mg, or about 100 mg.
Embodiment 103. The use of any one of Embodiments 101 to 102, wherein PRC2-mediated disease or disorder is diffused large B cell lymphoma (DLBCL), follicular lymphoma, leukemia, multiple myeloma, mesothelioma, gastric cancer, malignant rhabdoid tumor, hepatocellular carcinoma, prostate cancer, breast carcinoma, bile duct cancer, gallbladder cancers, bladder carcinoma, neuroblastoma, schwannoma, glioma, glioblastoma, astrocytoma, cervical cancer, colon cancer, melanoma, endometrial cancer, esophageal cancer, head and neck cancer, lung cancer, nasopharyngeal carcinoma, ovarian cancer, pancreatic cancer, renal cell carcinoma, rectal cancer, thyroid cancer, parathyroid tumor, uterine tumor, or soft tissue sarcoma.
Embodiment 104. The use of any one of Embodiments 101 to 103, wherein the PRC2-mediated disease or disorder is diffused large B cell lymphoma (DLBCL), gastric cancer, prostate cancer, nasopharyngeal carcinoma, ovarian cancer, or soft tissue sarcoma.

In another aspect, the present invention provides a medicament comprising N-((5-fluoro-2,3-dihydrobenzofuran-4-yl)methyl)-8-(2-methylpyridin-3-yl)-[1,2,4]triazolo[4,3-c]pyrimidin-5-amine, or a pharmaceutically acceptable salt thereof, for use in the treatment of a PRC2-mediated disease or disorder, wherein N-((5-fluoro-2,3-dihydrobenzofuran-4-yl)methyl)-8-(2-methylpyridin-3-yl)-[1,2,4]triazolo[4,3-c]pyrimidin-5-amine, is present in an amount of about 2.5 mg to about 100 mg.

Certain aspects and examples of such medicaments for the treatment of a PRC2-mediated disease or disorder, are provided in the following listing of enumerated embodiments. It will be recognized that features specified in each embodiment may be combined with other specified features to provide further embodiments of such medicaments.
Embodiment 105. A medicament comprising N-((5-fluoro-2,3-dihydrobenzofuran-4-yl)methyl)-8-(2-methylpyridin-3-yl)-[1,2,4]triazolo[4,3-c]pyrimidin-5-amine, or a pharmaceutically acceptable salt thereof, wherein N-((5-fluoro-2,3-dihydrobenzofuran-4-yl)methyl)-8-(2-methylpyridin-3-yl)-[1,2,4]triazolo[4,3-c]pyrimidin-5-amine is present in an amount of about 2.5 mg to about 100 mg.
Embodiment 106. The medicament of Embodiment 105, wherein N-((5-fluoro-2,3- dihydrobenzofuran-4-yl)methyl)-8-(2-methylpyridin-3-yl)-[1,2,4]triazolo[4,3-c]pyrimidin-5-amine is present in an amount of about 2.5 mg, about 10 mg, about 50 mg, or about 100 mg.
Embodiment 107. The medicament of Embodiment 105 or Embodiment 106, wherein the medicament is suitable for oral administration of up to a maximum total dose of 800 mg per day of N-((5-fluoro-2,3-dihydrobenzofuran-4yl)methyl)-8-(2-methylpyridin-3-yl)-[1,2,4]triazolo[4,3-c]pyrimidin-5-amine for the treatment of a PRC2-mediated disease or disorder.
Embodiment 108. The medicament of any one of Embodiments 105 to 107, wherein PRC2-mediated disease or disorder is diffused large B cell lymphoma (DLBCL), follicular lymphoma, leukemia, multiple myeloma, mesothelioma, gastric cancer, malignant rhabdoid tumor, hepatocellular carcinoma, prostate cancer, breast carcinoma, bile duct cancer, gallbladder cancers, bladder carcinoma, neuroblastoma, schwannoma, glioma, glioblastoma, astrocytoma, cervical cancer, colon cancer, melanoma, endometrial cancer, esophageal cancer, head and neck cancer, lung cancer, nasopharyngeal carcinoma, ovarian cancer, pancreatic cancer, renal cell carcinoma, rectal cancer, thyroid cancer, parathyroid tumor, uterine tumor, or soft tissue sarcoma.
Embodiment 109. The medicament of any one of Embodiments 105 to 108, wherein the PRC2-mediated disease or disorder is diffused large B cell lymphoma (DLBCL), gastric cancer, prostate cancer, nasopharyngeal carcinoma, ovarian cancer, or soft tissue sarcoma.

### Pharmacology and Utility

In another aspect, the present invention provides the use of the dosages, dosage forms, medicaments, pharmaceutical compositions and dosage regimens provided herein in the treatment a PRC2-mediated disease or disorder. The PRC2-mediated disease or disorder treatable using the dosage forms, medicaments, pharmaceutical compositions and dosage regimens provides herein is diffused large B cell lymphoma (DLBCL), follicular lymphoma, leukemia, multiple myeloma, mesothelioma, gastric cancer, malignant rhabdoid tumor, hepatocellular carcinoma, prostate cancer, breast carcinoma, bile duct cancer, gallbladder cancers, bladder carcinoma, neuroblastoma, schwannoma, glioma, glioblastoma, astrocytoma, cervical cancer, colon cancer, melanoma, endometrial cancer, esophageal cancer, head and neck cancer, lung cancer, nasopharyngeal carcinoma, ovarian cancer, pancreatic cancer, renal cell carcinoma, rectal cancer, thyroid cancer, parathyroid tumor, uterine tumor, or soft tissue sarcoma. In certain embodiments the PRC2-mediated disease or disorder treatable using the dosages, dosage forms, medicaments, pharmaceutical compositions and dosage regimens provides herein is diffused large B cell lymphoma (DLBCL), gastric cancer, prostate cancer, nasopharyngeal carcinoma, ovarian cancer, or soft tissue sarcoma.

Accordingly, the invention further provides a method for treating a PRC2-mediated disease or disorder in a subject in need thereof using dosages, dosage forms, medicaments, pharmaceutical compositions and dosage regimens provides herein, wherein the method comprises administering to the subject in need thereof N-((5-fluoro-2,3-dihydrobenzofuran-4-yl)methyl)-8-(2-methylpyridin-3-yl)-[1,2,4]triazolo[4,3-c]pyrimidin-5-amine (Compound (1)), or a pharmaceutically acceptable salt thereof,

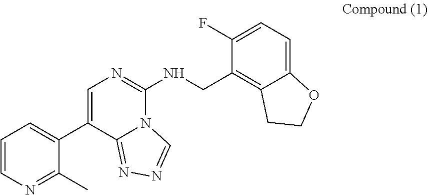 [SMILES: Cc1ncccc1-c1cnc(NCc2c(F)ccc3c2CCO3)n2cnnc12]

wherein Compound (1), or a pharmaceutically acceptable salt thereof is administered once a day at a dose of about 10 mg to about 800 mg, or Compound (1), or a pharmaceutically acceptable salt thereof is administered twice a day at a dose of about 60 mg to about 300 mg.

Certain aspects and examples of such methods using N-((5-fluoro-2,3-dihydrobenzofuran-4-yl)methyl)-8-(2-methylpyridin-3-yl)-[1,2,4]triazolo[4,3-c]pyrimidin-5-amine, or a pharmaceutically acceptable salt thereof, are provided in the following listing of enumerated embodiments. It is recognized that features specified in each embodiment may be combined with other specified features to provide further embodiments of such methods to treat PRC2-mediated diseases or disorders.
Embodiment 110. A method for treating a PRC2-mediated disease or disorder in a subject in need thereof comprising administering to the subject in need thereof a therapeutically effective amount of N-((5-fluoro-2,3-dihydrobenzofuran-4-yl)methyl)-8-(2-methylpyridin-3-yl)-[1,2,4]triazolo[4,3-c]pyrimidin-5-amine (Compound (1)), or a pharmaceutically acceptable salt thereof,

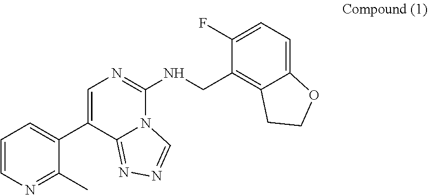 [SMILES: Cc1ncccc1-c1cnc(NCc2c(F)ccc3c2CCO3)n2cnnc12]

wherein Compound (1), or a pharmaceutically acceptable salt thereof, is administered daily.
Embodiment 111. A method for treating a PRC2-mediated disease or disorder in a subject in need thereof comprising administering to the subject in need thereof N-((5-fluoro-2,3-dihydrobenzofuran-4-yl)methyl)-8-(2-methylpyridin-3-yl)-[1,2,4]triazolo[4,3-c]pyrimidin-5-amine (Compound (1)), or a pharmaceutically acceptable salt thereof,

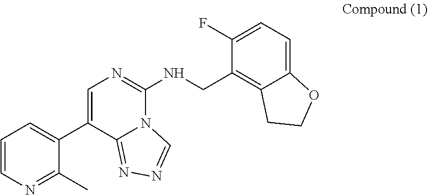 [SMILES: Cc1ncccc1-c1cnc(NCc2c(F)ccc3c2CCO3)n2cnnc12]

wherein Compound (1), or a pharmaceutically acceptable salt thereof, is administered daily.
Embodiment 112. The method of Embodiment 110 or Embodiment 111, wherein Compound (1) is administered as the hydrochloride salt.
Embodiment 113. The method of any one of Embodiments 110 to 112, wherein Compound (1), or a pharmaceutically acceptable salt thereof, is administered once a day.
Embodiment 114. The method of any one of Embodiments 110 to 113, wherein Compound (1), or a pharmaceutically acceptable salt thereof, is administered once a day at a dose of about 10 mg to about 800 mg.
Embodiment 115. The method of any one of Embodiments 108 to 114, wherein Compound (1), or pharmaceutically acceptable salt thereof, is administered once a day at a dose of about 10 mg, about 20 mg, about 40 mg, about 80 mg, about 120 mg, about 160 mg, about 240 mg, about 300 mg, about 500 mg or about 800 mg.
Embodiment 116. The method of any one of Embodiments 110 to 115, wherein Compound (1), or pharmaceutically acceptable salt thereof, is administered once a day at a dose of about 10 mg.
Embodiment 117. The method of any one of Embodiments 110 to 115, wherein Compound (1), or pharmaceutically acceptable salt thereof, is administered once a day at a dose of about 20 mg.
Embodiment 118. The method of any one of Embodiments 110 to 115, wherein Compound (1), or pharmaceutically acceptable salt thereof, is administered once a day at a dose of about 40 mg.
Embodiment 119. The method of any one of Embodiments 110 to 115, wherein Compound (1), or pharmaceutically acceptable salt thereof, is administered once a day at a dose of about 80 mg.
Embodiment 120. The method of any one of Embodiments 110 to 115, wherein Compound (1), or pharmaceutically acceptable salt thereof, is administered once a day at a dose of about 120 mg.
Embodiment 121. The method of any one of Embodiments 110 to 115, wherein Compound (1), or pharmaceutically acceptable salt thereof, is administered once a day at a dose of about 160 mg.
Embodiment 122. The method of any one of Embodiments 110 to 115, wherein Compound (1), or pharmaceutically acceptable salt thereof, is administered once a day at a dose of about 240 mg.
Embodiment 123. The method of any one of Embodiments 110 to 115, wherein Compound (1), or pharmaceutically acceptable salt thereof, is administered once a day at a dose of about 300 mg.
Embodiment 124. The method of any one of Embodiments 110 to 115, wherein Compound (1), or pharmaceutically acceptable salt thereof, is administered once a day at a dose of about 500 mg.
Embodiment 125. The method of any one of Embodiments 110 to 115, wherein Compound (1), or pharmaceutically acceptable salt thereof, is administered once a day at a dose of about 800 mg.
Embodiment 126. The method of any one of Embodiments 110 to 115, wherein Compound (1), or a pharmaceutically acceptable salt thereof, is administered once a day at a dose of about 10 mg to about 800 mg for eight consecutive days.
Embodiment 127. The method of any one of Embodiments 110 to 115, wherein Compound (1), or pharmaceutically acceptable salt thereof, is administered once a day at a dose of about 10 mg, about 20 mg, about 40 mg, about 80 mg, about 120 mg, about 160 mg, about 240 mg, about 300 mg, about 500 mg or about 800 mg for eight consecutive days.
Embodiment 128. The method of any one of Embodiments 110 to 115, wherein Compound (1), or pharmaceutically acceptable salt thereof, is administered once a day at a dose of about 10 mg for eight consecutive days.
Embodiment 129. The method of any one of Embodiments 110 to 115, wherein Compound (1), or pharmaceutically acceptable salt thereof, is administered once a day at a dose of about 20 mg for eight consecutive days.
Embodiment 130. The method of any one of Embodiments 110 to 115, wherein Compound (1), or pharmaceutically acceptable salt thereof, is administered once a day at a dose of about 40 mg for eight consecutive days.
Embodiment 131. The method of any one of Embodiments 110 to 115, wherein Compound (1), or pharmaceutically acceptable salt thereof, is administered once a day at a dose of about 80 mg for eight consecutive days.

Embodiment 132. The method of any one of Embodiments 110 to 115, wherein Compound (1), or pharmaceutically acceptable salt thereof, is administered once a day at a dose of about 120 mg for eight consecutive days.
Embodiment 133. The method of any one of Embodiments 110 to 115, wherein Compound (1), or pharmaceutically acceptable salt thereof, is administered once a day at a dose of about 160 mg for eight consecutive days.
Embodiment 134. The method of any one of Embodiments 110 to 115, wherein Compound (1), or pharmaceutically acceptable salt thereof, is administered once a day at a dose of about 240 mg for eight consecutive days.
Embodiment 135. The method of any one of Embodiments 110 to 115, wherein Compound (1), or pharmaceutically acceptable salt thereof, is administered once a day at a dose of about 300 mg for eight consecutive days.
Embodiment 136. The method of any one of Embodiments 110 to 115, wherein Compound (1), or pharmaceutically acceptable salt thereof, is administered once a day at a dose of about 500 mg for eight consecutive days.
Embodiment 137. The method of any one of Embodiments 110 to 115, wherein Compound (1), or pharmaceutically acceptable salt thereof, is administered once a day at a dose of about 800 mg for eight consecutive days.
Embodiment 138. The method of any one of Embodiments 110 to 112, wherein Compound (1), or a pharmaceutically acceptable salt thereof, is administered twice a day.
Embodiment 139. The method of any one of Embodiments 110 to 112 or Embodiment 136, wherein Compound (1), or a pharmaceutically acceptable salt thereof, is administered twice a day at a dose of about 60 mg to about 300 mg.
Embodiment 140. The method of any one of Embodiments 110 to 112 or Embodiments 136 to 137, wherein Compound (1), or a pharmaceutically acceptable salt thereof, is administered twice a day at a dose of about 60 mg, about 80 mg, about 120 mg, about 150 mg, or about 300 mg.
Embodiment 141. The method of any one of Embodiments 110 to 112 or Embodiments 136 to 137, wherein Compound (1), or pharmaceutically acceptable salt thereof, is administered twice a day at a dose of about 60 mg.
Embodiment 142. The method of any one of Embodiments 110 to 112 or Embodiments 136 to 137, wherein Compound (1), or pharmaceutically acceptable salt thereof, is administered twice a day at a dose of about 80 mg.
Embodiment 143. The method of any one of Embodiments 110 to 112 or Embodiments 136 to 137, wherein Compound (1), or pharmaceutically acceptable salt thereof, is administered twice a day at a dose of about 120 mg.
Embodiment 144. The method of any one of Embodiments 110 to 112 or Embodiments 136 to 137, wherein Compound (1), or pharmaceutically acceptable salt thereof, is administered twice a day at a dose of about 150 mg.
Embodiment 145. The method of any one of Embodiments 110 to 112 or Embodiments 136 to 137, wherein Compound (1), or pharmaceutically acceptable salt thereof, is administered twice a day at a dose of about 300 mg.
Embodiment 146. The method of any one of Embodiments 110 to 112, wherein Compound (1), or a pharmaceutically acceptable salt thereof, is administered twice a day for eight consecutive days.
Embodiment 147. The method of any one of Embodiments 110 to 112 or Embodiment 136, wherein Compound (1), or a pharmaceutically acceptable salt thereof, is administered twice a day at a dose of about 60 mg to about 300 mg for eight consecutive days.
Embodiment 148. The method of any one of Embodiments 110 to 112 or Embodiments 136 to 137, wherein Compound (1), or a pharmaceutically acceptable salt thereof, is administered twice a day at a dose of about 60 mg, about 80 mg, about 120 mg, about 150 mg, or about 300 mg for eight consecutive days.
Embodiment 149. The method of any one of Embodiments 110 to 112 or Embodiments 136 to 137, wherein Compound (1), or pharmaceutically acceptable salt thereof, is administered twice a day at a dose of about 60 mg for eight consecutive days.
Embodiment 150. The method of any one of Embodiments 110 to 112 or Embodiments 136 to 137, wherein Compound (1), or pharmaceutically acceptable salt thereof, is administered twice a day at a dose of about 80 mg for eight consecutive days.
Embodiment 151. The method of any one of Embodiments 110 to 112 or Embodiments 136 to 137, wherein Compound (1), or pharmaceutically acceptable salt thereof, is administered twice a day at a dose of about 120 mg for eight consecutive days.
Embodiment 152. The method of any one of Embodiments 110 to 112 or Embodiments 136 to 137, wherein Compound (1), or pharmaceutically acceptable salt thereof, is administered twice a day at a dose of about 150 mg for eight consecutive days.
Embodiment 153. The method of any one of Embodiments 110 to 112 or Embodiments 136 to 137, wherein Compound (1), or pharmaceutically acceptable salt thereof, is administered twice a day at a dose of about 300 mg for eight consecutive days.
Embodiment 154. The method of any one of Embodiments 110 to 153, wherein Compound (1), or a pharmaceutically acceptable salt thereof, is orally administered.
Embodiment 155. The method of any one of Embodiments 110 to 154, wherein Compound (1), or a pharmaceutically acceptable salt thereof, is administered as a continuous dosing regimen comprising one or more continuous dosing cycles.
Embodiment 156. The method of Embodiment 155, wherein the continuous dosing regimen comprises two continuous dosing cycles.
Embodiment 157. The method of Embodiment 155 or Embodiment 156, wherein each continuous dosing cycle is an eight day cycle.
Embodiment 158. The method of any one of Embodiments 155 to 157, wherein Compound (1), or a pharmaceutically acceptable salt thereof, is administered as a cyclical dosing regimen comprising one or more continuous dosing cycles and one or more dose delays.
Embodiment 159. The method of Embodiment 158, wherein the cyclical dosing regimen comprising two continuous dosing cycles and one dose delay.

Embodiment 160. The method of Embodiment 158 or Embodiment 159, wherein each continuous dosing cycle is an eight day cycle and the dose delay is eight or more days.
Embodiment 161. The method of Embodiment 158 or Embodiment 159, wherein each continuous dosing cycle is an eight day cycle and the dose delay is eight days.
Embodiment 162. The method of any one of Embodiments 110 to 161, wherein PRC2-mediated disease or disorder is diffused large B cell lymphoma (DLBCL), follicular lymphoma, leukemia, multiple myeloma, mesothelioma, gastric cancer, malignant rhabdoid tumor, hepatocellular carcinoma, prostate cancer, breast carcinoma, bile duct cancer, gallbladder cancers, bladder carcinoma, neuroblastoma, schwannoma, glioma, glioblastoma, astrocytoma, cervical cancer, colon cancer, melanoma, endometrial cancer, esophageal cancer, head and neck cancer, lung cancer, nasopharyngeal carcinoma, ovarian cancer, pancreatic cancer, renal cell carcinoma, rectal cancer, thyroid cancer, parathyroid tumor, uterine tumor, or soft tissue sarcoma.
Embodiment 163. The method of any one of Embodiments 110 to 162, wherein the PRC2-mediated disease or disorder is diffused large B cell lymphoma (DLBCL), gastric cancer, prostate cancer, nasopharyngeal carcinoma, ovarian cancer, or soft tissue sarcoma.
Embodiment 164. A method for treating a PRC2-mediated disease or disorder in a subject in need thereof comprising administering to the subject in need thereof a therapeutically effective amount of N-((5-fluoro-2,3-dihydrobenzofuran-4-yl)methyl)-8-(2-methylpyridin-3-yl)-[1,2,4]triazolo[4,3-c]pyrimidin-5-amine (Compound (1)), or a pharmaceutically acceptable salt thereof,

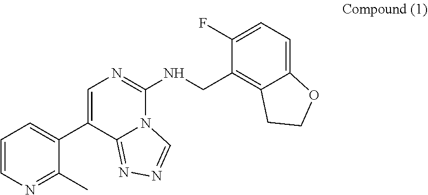 [SMILES: Cc1ncccc1-c1cnc(NCc2c(F)ccc3c2CCO3)n2cnnc12]

wherein Compound (1), or a pharmaceutically acceptable salt thereof, is administered once a day in an amount sufficient to provide Compound (1) at a dose of about 10 mg to about 800 mg, or Compound (1), or a pharmaceutically acceptable salt thereof, is administered twice a day in an amount sufficient to provide Compound (1) at a dose of about 60 mg to about 300 mg.
Embodiment 165. A method for treating a PRC2-mediated disease or disorder in a subject in need thereof comprising administering to the subject in need thereof N-((5-fluoro-2,3-dihydrobenzofuran-4-yl)methyl)-8-(2-methylpyridin-3-yl)-[1,2,4]triazolo[4,3-c]pyrimidin-5-amine (Compound (1)), or a pharmaceutically acceptable salt thereof,

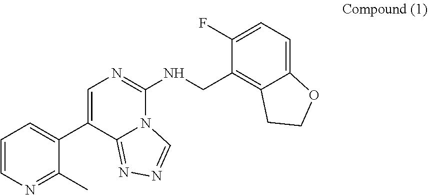 [SMILES: Cc1ncccc1-c1cnc(NCc2c(F)ccc3c2CCO3)n2cnnc12]

wherein Compound (1), or a pharmaceutically acceptable salt thereof, is administered once a day in an amount sufficient to provide Compound (1) at a dose of about 10 mg to about 800 mg, or Compound (1), or a pharmaceutically acceptable salt thereof, is administered twice a day in an amount sufficient to provide Compound (1) at a dose of about 60 mg to about 300 mg.
Embodiment 166. The method of Embodiment 164 or Embodiment 165, wherein Compound (1), or a pharmaceutically acceptable salt thereof, is administered once a day in an amount sufficient to provide Compound (1) at a dose of about 10 mg to about 800 mg.
Embodiment 167. The method of any one of Embodiments 164 to 166, wherein Compound (1), or pharmaceutically acceptable salt thereof, is administered once a day in an amount sufficient to provide Compound (1) at a dose of about 10 mg, about 20 mg, about 40 mg, about 80 mg, about 120 mg, about 160 mg, about 240 mg, about 300 mg, about 500 mg or about 800 mg.
Embodiment 168. The method of any one of Embodiments 164 to 166, wherein Compound (1), or pharmaceutically acceptable salt thereof, is administered once a day in an amount sufficient to provide Compound (1) at a dose of about 10 mg.
Embodiment 169. The method of any one of Embodiments 164 to 166, wherein Compound (1), or pharmaceutically acceptable salt thereof, is administered once a day in an amount sufficient to provide Compound (1) at a dose of about 20 mg.
Embodiment 170. The method of any one of Embodiments 164 to 166, wherein Compound (1), or pharmaceutically acceptable salt thereof, is administered once a day in an amount sufficient to provide Compound (1) at a dose of about 40 mg.
Embodiment 171. The method of any one of Embodiments 164 to 166, wherein Compound (1), or pharmaceutically acceptable salt thereof, is administered once a day in an amount sufficient to provide Compound (1) at a dose of about 80 mg.
Embodiment 172. The method of any one of Embodiments 164 to 166, wherein Compound (1), or pharmaceutically acceptable salt thereof, is administered once a day in an amount sufficient to provide Compound (1) at a dose of about 120 mg.
Embodiment 173. The method of any one of Embodiments 164 to 166, wherein Compound (1), or pharmaceutically acceptable salt thereof, is administered once a day in an amount sufficient to provide Compound (1) at a dose of about 160 mg.
Embodiment 174. The method of any one of Embodiments 164 to 166, wherein Compound (1), or pharmaceutically acceptable salt thereof, is administered once a day in an amount sufficient to provide Compound (1) at a dose of about 240 mg.
Embodiment 175. The method of any one of Embodiments 164 to 166, wherein Compound (1), or pharmaceutically acceptable salt thereof, is administered once a day in an amount sufficient to provide Compound (1) at a dose of about 300 mg.
Embodiment 176. The method of any one of Embodiments 164 to 166, wherein Compound (1), or pharmaceutically acceptable salt thereof, is administered once a day in an amount sufficient to provide Compound (1) at a dose of about 500 mg.
Embodiment 177. The method of any one of Embodiments 164 to 166, wherein Compound (1), or pharmaceutically acceptable salt thereof, is administered once a day in an amount sufficient to provide Compound (1) at a dose of about 800 mg.
Embodiment 178. The method of Embodiment 164 or Embodiment 165, wherein Compound (1), or a pharmaceutically acceptable salt thereof, is administered twice a day in an amount sufficient to provide Compound (1) at a dose of about 60 mg to about 300 mg.
Embodiment 179. The method of any one of Embodiments 164 to 165 or Embodiment 178, wherein Compound (1), or a pharmaceutically acceptable salt thereof, is administered twice a day in an amount sufficient to provide Compound (1) at a dose of about 60 mg, about 80 mg, about 120 mg, about 150 mg, or about 300 mg.
Embodiment 180. The method of any one of Embodiments 164 to 165 or Embodiments 178 to 179, wherein Compound (1), or pharmaceutically acceptable salt thereof, is administered twice a day in an amount sufficient to provide Compound (1) at a dose of about 60 mg.
Embodiment 181. The method of any one of Embodiments 164 to 165 or Embodiments 178 to 179, wherein Compound (1), or pharmaceutically acceptable salt thereof, is administered twice a day in an amount sufficient to provide Compound (1) at a dose of about 80 mg.
Embodiment 182. The method of any one of Embodiments 164 to 165 or Embodiments 178 to 179, wherein Compound (1), or pharmaceutically acceptable salt thereof, is administered twice a day in an amount sufficient to provide Compound (1) at a dose of about 120 mg.
Embodiment 183. The method of any one of Embodiments 164 to 165 or Embodiments 178 to 179, wherein Compound (1), or pharmaceutically acceptable salt thereof, is administered twice a day in an amount sufficient to provide Compound (1) at a dose of about 150 mg.
Embodiment 184. The method of any one of Embodiments 164 to 165 or Embodiments 178 to 179, wherein Compound (1), or pharmaceutically acceptable salt thereof, is administered twice a day in an amount sufficient to provide Compound (1) at a dose of about 300 mg.
Embodiment 185. The method of any one of Embodiments 164 to 184, wherein Compound (1), or a pharmaceutically acceptable salt thereof, is orally administered.
Embodiment 186. The method of any one of Embodiments 164 to 185, wherein PRC2-mediated disease or disorder is diffused large B cell lymphoma (DLBCL), follicular lymphoma, leukemia, multiple myeloma, mesothelioma, gastric cancer, malignant rhabdoid tumor, hepatocellular carcinoma, prostate cancer, breast carcinoma, bile duct cancer, gallbladder cancers, bladder carcinoma, neuroblastoma, schwannoma, glioma, glioblastoma, astrocytoma, cervical cancer, colon cancer, melanoma, endometrial cancer, esophageal cancer, head and neck cancer, lung cancer, nasopharyngeal carcinoma, ovarian cancer, pancreatic cancer, renal cell carcinoma, rectal cancer, thyroid cancer, parathyroid tumor, uterine tumor, or soft tissue sarcoma.
Embodiment 187. The method of any one of Embodiments 164 to 186, wherein the PRC2-mediated disease or disorder is diffused large B cell lymphoma (DLBCL), gastric cancer, prostate cancer, nasopharyngeal carcinoma, ovarian cancer, or soft tissue sarcoma.

The invention further provides the use of Compound (1), or a pharmaceutically acceptable salt thereof, for the treatment of a PRC2-mediated disease or disorder, wherein Compound (1), or a pharmaceutically acceptable salt thereof, is used once a day at a dose of about 10 mg to about 800 mg, or Compound (1), or a pharmaceutically acceptable salt thereof, is used twice a day at a dose of about 60 mg to about 300 mg.

Certain aspects and examples of such uses of Compound (1), or a pharmaceutically acceptable salt thereof, are provided in the following listing of enumerated embodiments. It is recognized that features specified in each embodiment may be combined with other specified features to provide further embodiments of such uses to treat PRC2-mediated diseases or disorders.
Embodiment 188. Use of Compound (1), or a pharmaceutically acceptable salt thereof, for the treatment of a PRC2-mediated disease or disorder, wherein Compound (1), or a pharmaceutically acceptable salt thereof, is used daily.
Embodiment 189. The use of Embodiment 188, wherein Compound (1) is used as the hydrochloride salt.
Embodiment 190. The use of any one of Embodiments 188 to 189, wherein Compound (1), or a pharmaceutically acceptable salt thereof, is used once a day at a dose of about 10 mg to about 800 mg or Compound (1), or a pharmaceutically acceptable salt thereof, is used twice a day at a dose of about 60 mg to about 300 mg.
Embodiment 191. The use of any one of Embodiments 186 to 190, wherein Compound (1), or a pharmaceutically acceptable salt thereof, is used once a day.
Embodiment 192. The use of any one of Embodiments 188 to 190, wherein Compound (1), or a pharmaceutically acceptable salt thereof, is used once a day at a dose of about 10 mg to about 800 mg.
Embodiment 193. The use of any one of Embodiments 188 to 190, wherein Compound (1), or pharmaceutically acceptable salt thereof, is used once a day at a dose of about 10 mg, about 20 mg, about 40 mg, about 80 mg, about 120 mg, about 160 mg, about 240 mg, about 300 mg, about 500 mg or about 800 mg.
Embodiment 194. The use of any one of Embodiments 188 to 190, wherein Compound (1), or pharmaceutically acceptable salt thereof, is used once a day at a dose of about 10 mg.
Embodiment 195. The use of any one of Embodiments 188 to 190, wherein Compound (1), or pharmaceutically acceptable salt thereof, is used once a day at a dose of about 20 mg.
Embodiment 196. The use of any one of Embodiments 188 to 190, wherein Compound (1), or pharmaceutically acceptable salt thereof, is used once a day at a dose of about 40 mg.
Embodiment 197. The use of any one of Embodiments 188 to 190, wherein Compound (1), or pharmaceutically acceptable salt thereof, is used once a day at a dose of about 80 mg.
Embodiment 198. The use of any one of Embodiments 188 to 190, wherein Compound (1), or pharmaceutically acceptable salt thereof, is used once a day at a dose of about 120 mg.
Embodiment 199. The use of any one of Embodiments 188 to 190, wherein Compound (1), or pharmaceutically acceptable salt thereof, is used once a day at a dose of about 160 mg.
Embodiment 200. The use of any one of Embodiments 188 to 190, wherein Compound (1), or pharmaceutically acceptable salt thereof, is used once a day at a dose of about 240 mg.
Embodiment 201. The use of any one of Embodiments 188 to 190, wherein Compound (1), or pharmaceutically acceptable salt thereof, is used once a day at a dose of about 300 mg.
Embodiment 202. The use of any one of Embodiments 188 to 190, wherein Compound (1), or pharmaceutically acceptable salt thereof, is used once a day at a dose of about 500 mg.
Embodiment 203. The use of any one of Embodiments 188 to 190, wherein Compound (1), or pharmaceutically acceptable salt thereof, is used once a day at a dose of about 800 mg.
Embodiment 204. The use of any one of Embodiments 188 to 190, wherein Compound (1), or a pharmaceutically acceptable salt thereof, is used once a day at a dose of about 10 mg to about 800 mg for eight consecutive days.
Embodiment 205. The use of any one of Embodiments 188 to 190, wherein Compound (1), or pharmaceutically acceptable salt thereof, is used once a day at a dose of about 10 mg, about 20 mg, about 40 mg, about 80 mg, about 120 mg, about 160 mg, about 240 mg, about 300 mg, about 500 mg or about 800 mg for eight consecutive days.
Embodiment 206. The use of any one of Embodiments 188 to 190, wherein Compound (1), or pharmaceutically acceptable salt thereof, is used once a day at a dose of about 10 mg for eight consecutive days.
Embodiment 207. The use of any one of Embodiments 188 to 190, wherein Compound (1), or pharmaceutically acceptable salt thereof, is used once a day at a dose of about 20 mg for eight consecutive days.
Embodiment 208. The use of any one of Embodiments 188 to 190, wherein Compound (1), or pharmaceutically acceptable salt thereof, is used once a day at a dose of about 40 mg for eight consecutive days.
Embodiment 209. The use of any one of Embodiments 188 to 190, wherein Compound (1), or pharmaceutically acceptable salt thereof, is used once a day at a dose of about 80 mg for eight consecutive days.
Embodiment 210. The use of any one of Embodiments 188 to 190, wherein Compound (1), or pharmaceutically acceptable salt thereof, is used once a day at a dose of about 120 mg for eight consecutive days.
Embodiment 211. The use of any one of Embodiments 188 to 190, wherein Compound (1), or pharmaceutically acceptable salt thereof, is used once a day at a dose of about 160 mg for eight consecutive days.
Embodiment 212. The use of any one of Embodiments 188 to 190, wherein Compound (1), or pharmaceutically acceptable salt thereof, is used once a day at a dose of about 240 mg for eight consecutive days.
Embodiment 213. The use of any one of Embodiments 188 to 190, wherein Compound (1), or pharmaceutically acceptable salt thereof, is used once a day at a dose of about 300 mg for eight consecutive days.
Embodiment 214. The use of any one of Embodiments 188 to 190, wherein Compound (1), or pharmaceutically acceptable salt thereof, is used once a day at a dose of about 500 mg for eight consecutive days.
Embodiment 215. The use of any one of Embodiments 188 to 190, wherein Compound (1), or pharmaceutically acceptable salt thereof, is used once a day at a dose of about 800 mg for eight consecutive days.
Embodiment 216. The use of any one of Embodiments 188 to 188, wherein Compound (1), or a pharmaceutically acceptable salt thereof, is used twice a day.
Embodiment 217. The use of any one of Embodiments 188 to 190 or Embodiment 214, wherein Compound (1), or a pharmaceutically acceptable salt thereof, is used twice a day at a dose of about 60 mg to about 300 mg.
Embodiment 218. The use of any one of Embodiments 188 to 190 or any one of Embodiments 214 to 215, wherein Compound (1), or pharmaceutically acceptable salt thereof, is used twice a day at a dose of about 60 mg, about 80 mg, about 120 mg, about 150 mg, or about 300 mg.
Embodiment 219. The use of any one of Embodiments 188 to 190 or any one of Embodiments 214 to 216, wherein Compound (1), or pharmaceutically acceptable salt thereof, is used twice a day at a dose of about 60 mg.

Embodiment 220. The use of any one of Embodiments 188 to 190 or any one of Embodiments 214 to 216, wherein Compound (1), or pharmaceutically acceptable salt thereof, is used twice a day at a dose of about 80 mg.
Embodiment 221. The use of any one of Embodiments 188 to 190 or any one of Embodiments 214 to 216, wherein Compound (1), or pharmaceutically acceptable salt thereof, is used twice a day at a dose of about 120 mg.
Embodiment 222. The use of any one of Embodiments 188 to 190 or any one of Embodiments 214 to 216, wherein Compound (1), or pharmaceutically acceptable salt thereof, is used twice a day at a dose of about 150 mg.
Embodiment 223. The use of any one of Embodiments 188 to 190 or any one of Embodiments 214 to 216, wherein Compound (1), or pharmaceutically acceptable salt thereof, is used twice a day at a dose of about 300 mg.
Embodiment 224. The use of any one of Embodiments 188 to 190 or Embodiment 214, wherein Compound (1), or a pharmaceutically acceptable salt thereof, is used twice a day at a dose of about 60 mg to about 300 mg for eight consecutive days.
Embodiment 225. The use of any one of Embodiments 188 to 190 or any one of Embodiments 214 to 215, wherein Compound (1), or pharmaceutically acceptable salt thereof, is used twice a day at a dose of about 60 mg, about 80 mg, about 120 mg, about 150 mg, or about 300 mg for eight consecutive days.
Embodiment 226. The use of any one of Embodiments 188 to 190 or any one of Embodiments 214 to 216, wherein Compound (1), or pharmaceutically acceptable salt thereof, is used twice a day at a dose of about 60 mg for eight consecutive days.
Embodiment 227. The use of any one of Embodiments 188 to 190 or any one of Embodiments 214 to 216, wherein Compound (1), or pharmaceutically acceptable salt thereof, is used twice a day at a dose of about 80 mg for eight consecutive days.
Embodiment 228. The use of any one of Embodiments 188 to 190 or any one of Embodiments 214 to 216, wherein Compound (1), or pharmaceutically acceptable salt thereof, is used twice a day at a dose of about 120 mg for eight consecutive days.
Embodiment 229. The use of any one of Embodiments 188 to 190 or any one of Embodiments 214 to 216, wherein Compound (1), or pharmaceutically acceptable salt thereof, is used twice a day at a dose of about 150 mg for eight consecutive days.
Embodiment 230. The use of any one of Embodiments 188 to 190 or any one of Embodiments 214 to 216, wherein Compound (1), or pharmaceutically acceptable salt thereof, is used twice a day at a dose of about 300 mg for eight consecutive days.
Embodiment 231. The use of any one of Embodiments 188 to 230, wherein PRC2-mediated disease or disorder is diffused large B cell lymphoma (DLBCL), follicular lymphoma, leukemia, multiple myeloma, mesothelioma, gastric cancer, malignant rhabdoid tumor, hepatocellular carcinoma, prostate cancer, breast carcinoma, bile duct cancer, gallbladder cancers, bladder carcinoma, neuroblastoma, schwannoma, glioma, glioblastoma, astrocytoma, cervical cancer, colon cancer, melanoma, endometrial cancer, esophageal cancer, head and neck cancer, lung cancer, nasopharyngeal carcinoma, ovarian cancer, pancreatic cancer, renal cell carcinoma, rectal cancer, thyroid cancer, parathyroid tumor, uterine tumor, or soft tissue sarcoma.
Embodiment 232. The method of any one of Embodiments 188 to 231, wherein the PRC2-mediated disease or disorder is diffused large B cell lymphoma (DLBCL), gastric cancer, prostate cancer, nasopharyngeal carcinoma, ovarian cancer, or soft tissue sarcoma.
Embodiment 233. Use of compound (1), or a pharmaceutically acceptable salt thereof, for the treatment of a PRC2-mediated disease or disorder, wherein Compound (1), or a pharmaceutically acceptable salt thereof, is used once a day in an amount sufficient to provide Compound (1) at a dose of about 10 mg to about 800 mg or Compound (1), or a pharmaceutically acceptable salt thereof, is used twice a day in an amount sufficient to provide Compound (1) at a dose of about 60 mg to about 300 mg.
Embodiment 234. The use of Embodiment 233, wherein Compound (1), or a pharmaceutically acceptable salt thereof, is used once a day in an amount sufficient to provide Compound (1) at a dose of about 10 mg to about 800 mg.
Embodiment 235. The use of Embodiment 233 or Embodiment 234, wherein Compound (1), or pharmaceutically acceptable salt thereof, is used once a day in an amount sufficient to provide Compound (1) at a dose of about 10 mg, about 20 mg, about 40 mg, about 80 mg, about 120 mg, about 160 mg, about 240 mg, about 300 mg, about 500 mg or about 800 mg.
Embodiment 236. The use of any one of Embodiments 233 to 234, wherein Compound (1), or pharmaceutically acceptable salt thereof, is used once a day in an amount sufficient to provide Compound (1) at a dose of about 10 mg.
Embodiment 237. The use of any one of Embodiments 233 to 234, wherein Compound (1), or pharmaceutically acceptable salt thereof, is used once a day in an amount sufficient to provide Compound (1) at a dose of about 20 mg.
Embodiment 238. The use of any one of Embodiments 233 to 234, wherein Compound (1), or pharmaceutically acceptable salt thereof, is used once a day in an amount sufficient to provide Compound (1) at a dose of about 40 mg.
Embodiment 239. The use of any one of Embodiments 233 to 234, wherein Compound (1), or pharmaceutically acceptable salt thereof, is used once a day in an amount sufficient to provide Compound (1) at a dose of about 80 mg.
Embodiment 240. The use of any one of Embodiments 233 to 234, wherein Compound (1), or pharmaceutically acceptable salt thereof, is used once a day in an amount sufficient to provide Compound (1) at a dose of about 120 mg.
Embodiment 241. The use of any one of Embodiments 233 to 234, wherein Compound (1), or pharmaceutically acceptable salt thereof, is used once a day in an amount sufficient to provide Compound (1) at a dose of about 160 mg.
Embodiment 242. The use of any one of Embodiments 233 to 234, wherein Compound (1), or pharmaceutically acceptable salt thereof, is used once a day in an amount sufficient to provide Compound (1) at a dose of about 240 mg.
Embodiment 243. The use of any one of Embodiments 233 to 234, wherein Compound (1), or pharmaceutically acceptable salt thereof, is used once a day in an amount sufficient to provide Compound (1) at a dose of about 300 mg.
Embodiment 244. The use of any one of Embodiments 233 to 234, wherein Compound (1), or pharmaceutically acceptable salt thereof, is used once a day in an amount sufficient to provide Compound (1) at a dose of about 500 mg.
Embodiment 245. The use of any one of Embodiments 233 to 234, wherein Compound (1), or pharmaceutically acceptable salt thereof, is used once a day in an amount sufficient to provide Compound (1) at a dose of about 800 mg.
Embodiment 246. The use of embodiment 233, wherein Compound (1), or a pharmaceutically acceptable salt thereof, is used twice a day in an amount sufficient to provide Compound (1) at a dose of about 60 mg to about 300 mg.
Embodiment 247. The use of Embodiment 233 or Embodiment 246, wherein Compound (1), or pharmaceutically acceptable salt thereof, is used twice a day in an amount sufficient to provide Compound (1) at a dose of about 60 mg, about 80 mg, about 120 mg, about 150 mg, or about 300 mg.
Embodiment 248. The use of Embodiment 233 or any one of Embodiments 246 to 247, wherein Compound (1), or pharmaceutically acceptable salt thereof, is used twice a day in an amount sufficient to provide Compound (1) at a dose of about 60 mg.
Embodiment 249. The use of Embodiment 233 or any one of Embodiments 246 to 247, wherein Compound (1), or pharmaceutically acceptable salt thereof, is used twice a day in an amount sufficient to provide Compound (1) at a dose of about 80 mg.
Embodiment 250. The use of Embodiment 233 or any one of Embodiments 246 to 247, wherein Compound (1), or pharmaceutically acceptable salt thereof, is used twice a day in an amount sufficient to provide Compound (1) at a dose of about 120 mg.
Embodiment 251. The use of Embodiment 233 or any one of Embodiments 246 to 247, wherein Compound (1), or pharmaceutically acceptable salt thereof, is used twice a day in an amount sufficient to provide Compound (1) at a dose of about 150 mg.
Embodiment 252. The use of Embodiment 233 or any one of Embodiments 246 to 247, wherein Compound (1), or pharmaceutically acceptable salt thereof, is used twice a day in an amount sufficient to provide Compound (1) at a dose of about 300 mg.
Embodiment 253. The use of any one of Embodiments 233 to 252, wherein PRC2-mediated disease or disorder is diffused large B cell lymphoma (DLBCL), follicular lymphoma, leukemia, multiple myeloma, mesothelioma, gastric cancer, malignant rhabdoid tumor, hepatocellular carcinoma, prostate cancer, breast carcinoma, bile duct cancer, gallbladder cancers, bladder carcinoma, neuroblastoma, schwannoma, glioma, glioblastoma, astrocytoma, cervical cancer, colon cancer, melanoma, endometrial cancer, esophageal cancer, head and neck cancer, lung cancer, nasopharyngeal carcinoma, ovarian cancer, pancreatic cancer, renal cell carcinoma, rectal cancer, thyroid cancer, parathyroid tumor, uterine tumor, or soft tissue sarcoma.
Embodiment 254. The use of any one of Embodiments 233 to 253, wherein the PRC2-mediated disease or disorder is diffused large B cell lymphoma (DLBCL), gastric cancer, prostate cancer, nasopharyngeal carcinoma, ovarian cancer, or soft tissue sarcoma.

The invention further provides Compound (1), or a pharmaceutically acceptable salt thereof, for the use in the treatment of a PRC2-mediated disease or disorder, wherein Compound (1), or a pharmaceutically acceptable salt thereof, is used once a day at a dose of about 10 mg to about 800 mg, or Compound (1), or a pharmaceutically acceptable salt thereof, is used twice a day at a dose of about 60 mg to about 300 mg.

Certain aspects and examples of such uses of Compound (1), or a pharmaceutically acceptable salt thereof, are provided in the following listing of enumerated embodiments. It is recognized that features specified in each embodiment may be combined with other specified features to provide further embodiments of such uses to treat PRC2-mediated diseases or disorders.
Embodiment 255. Compound (1), or a pharmaceutically acceptable salt thereof, for use in the treatment of a PRC2-mediated disease or disorder, wherein Compound (1),or a pharmaceutically acceptable salt thereof, is used daily.
Embodiment 256. The compound of Embodiment 255, wherein Compound (1) is used as the hydrochloride salt.
Embodiment 257. The compound of Embodiment 255 or Embodiment 256, wherein Compound (1),or a pharmaceutically acceptable salt thereof, is used once a day at a dose of about 10 mg to about 800 mg, or Compound (1), or a pharmaceutically acceptable salt thereof is used twice a day at a dose of about 60 mg to about 300 mg.
Embodiment 258. The compound of any one of Embodiments 255 to 257, wherein Compound (1), or a pharmaceutically acceptable salt thereof, is used once a day at a dose of about 10 mg to about 800 mg.
Embodiment 259. The compound of any one of Embodiments 255 to 257, wherein Compound (1), or pharmaceutically acceptable salt thereof, is used once a day at a dose of about 10 mg, about 20 mg, about 40 mg, about 80 mg, about 120 mg, about 160 mg, about 240 mg, about 300 mg, about 500 mg or about 800 mg.
Embodiment 260. The compound of any one of Embodiments 255 to 257, wherein Compound (1), or pharmaceutically acceptable salt thereof, is used once a day at a dose of about 10 mg.
Embodiment 261. The compound of any one of Embodiments 255 to 257, wherein Compound (1), or pharmaceutically acceptable salt thereof, is used once a day at a dose of about 20 mg.
Embodiment 262. The compound of any one of Embodiments 255 to 257, wherein Compound (1), or pharmaceutically acceptable salt thereof, is used once a day at a dose of about 40 mg.
Embodiment 263. The compound of any one of Embodiments 255 to 257, wherein Compound (1), or pharmaceutically acceptable salt thereof, is used once a day at a dose of about 80 mg.
Embodiment 264. The compound of any one of Embodiments 255 to 257, wherein Compound (1), or pharmaceutically acceptable salt thereof, is used once a day at a dose of about 120 mg.
Embodiment 265. The compound of any one of Embodiments 255 to 257, wherein Compound (1), or pharmaceutically acceptable salt thereof, is used once a day at a dose of about 160 mg.
Embodiment 266. The compound of any one of Embodiments 255 to 257, wherein Compound (1), or pharmaceutically acceptable salt thereof, is used once a day at a dose of about 240 mg.
Embodiment 267. The compound of any one of Embodiments 255 to 257, wherein Compound (1), or pharmaceutically acceptable salt thereof, is used once a day at a dose of about 300 mg.
Embodiment 268. The compound of any one of Embodiments 255 to 257, wherein Compound (1), or pharmaceutically acceptable salt thereof, is used once a day at a dose of about 500 mg.
Embodiment 269. The compound of any one of Embodiments 255 to 257, wherein Compound (1), or pharmaceutically acceptable salt thereof, is used once a day at a dose of about 800 mg.
Embodiment 270. The compound of any one of Embodiments 255 to 257, wherein Compound (1), or a pharmaceutically acceptable salt thereof, is used once a day at a dose of about 10 mg to about 800 mg for eight consecutive days.
Embodiment 271. The compound of any one of Embodiments 255 to 257, wherein Compound (1), or pharmaceutically acceptable salt thereof, is used once a day at a dose of about 10 mg, about 20 mg, about 40 mg, about 80 mg, about 120 mg, about 160 mg, about 240 mg, about 300 mg, about 500 mg or about 800 mg for eight consecutive days.
Embodiment 272. The compound of any one of Embodiments 255 to 257, wherein Compound (1), or pharmaceutically acceptable salt thereof, is used once a day at a dose of about 10 mg for eight consecutive days.
Embodiment 273. The compound of any one of Embodiments 255 to 257, wherein Compound (1), or pharmaceutically acceptable salt thereof, is used once a day at a dose of about 20 mg for eight consecutive days.
Embodiment 274. The compound of any one of Embodiments 255 to 257, wherein Compound (1), or pharmaceutically acceptable salt thereof, is used once a day at a dose of about 40 mg for eight consecutive days.
Embodiment 275. The compound of any one of Embodiments 255 to 257, wherein Compound (1), or pharmaceutically acceptable salt thereof, is used once a day at a dose of about 80 mg for eight consecutive days.
Embodiment 276. The compound of any one of Embodiments 255 to 257, wherein Compound (1), or pharmaceutically acceptable salt thereof, is used once a day at a dose of about 120 mg for eight consecutive days.
Embodiment 277. The compound of any one of Embodiments 255 to 257, wherein Compound (1), or pharmaceutically acceptable salt thereof, is used once a day at a dose of about 160 mg for eight consecutive days.
Embodiment 278. The compound of any one of Embodiments 255 to 257, wherein Compound (1), or pharmaceutically acceptable salt thereof, is used once a day at a dose of about 240 mg for eight consecutive days.
Embodiment 279. The compound of any one of Embodiments 255 to 257, wherein Compound (1), or pharmaceutically acceptable salt thereof, is used once a day at a dose of about 300 mg for eight consecutive days.
Embodiment 280. The compound of any one of Embodiments 255 to 257, wherein Compound (1), or pharmaceutically acceptable salt thereof, is used once a day at a dose of about 500 mg for eight consecutive days.
Embodiment 281. The compound of any one of Embodiments 255 to 257, wherein Compound (1), or pharmaceutically acceptable salt thereof, is used once a day at a dose of about 800 mg for eight consecutive days.
Embodiment 282. The compound of any one of Embodiments 255 to 257, wherein Compound (1), or a pharmaceutically acceptable salt thereof, is used twice a day at a dose of about 60 mg to about 300 mg.
Embodiment 283. The compound of any one of Embodiments 255 to 257 or Embodiment 288, wherein Compound (1), or pharmaceutically acceptable salt thereof, is used twice a day at a dose of about 60 mg, about 80 mg, about 120 mg, about 150 mg, or about 300 mg.
Embodiment 284. The compound of any one of Embodiments 255 to 257 or any one of Embodiments 288 to 289, wherein Compound (1), or pharmaceutically acceptable salt thereof, is used twice a day at a dose of about 60 mg.
Embodiment 285. The compound of any one of Embodiments 255 to 257 or any one of Embodiments 288 to 289, wherein Compound (1), or pharmaceutically acceptable salt thereof, is used twice a day at a dose of about 80 mg.
Embodiment 286. The compound of any one of Embodiments 255 to 257 or any one of Embodiments 288 to 289, wherein Compound (1), or pharmaceutically acceptable salt thereof, is used twice a day at a dose of about 120 mg.
Embodiment 287. The compound of any one of Embodiments 255 to 257 or any one of Embodiments 288 to 289, wherein Compound (1), or pharmaceutically acceptable salt thereof, is used twice a day at a dose of about 150 mg.
Embodiment 288. The compound of any one of Embodiments 255 to 257 or any one of Embodiments 288 to 289, wherein Compound (1), or pharmaceutically acceptable salt thereof, is used twice a day at a dose of about 300 mg.
Embodiment 289. The compound of any one of Embodiments 255 to 257, wherein Compound (1), or a pharmaceutically acceptable salt thereof, is used twice a day at a dose of about 60 mg to about 300 mg for eight consecutive days.
Embodiment 290. The compound of any one of Embodiments 255 to 257 or Embodiment 288, wherein Compound (1), or pharmaceutically acceptable salt thereof, is used twice a day at a dose of about 60 mg, about 80 mg, about 120 mg, about 150 mg, or about 300 mg for eight consecutive day.
Embodiment 291. The compound of any one of Embodiments 255 to 257 or any one of Embodiments 288 to 289, wherein Compound (1), or pharmaceutically acceptable salt thereof, is used twice a day at a dose of about 60 mg for eight consecutive day.
Embodiment 292. The compound of any one of Embodiments 255 to 257 or any one of Embodiments 288 to 289, wherein Compound (1), or pharmaceutically acceptable salt thereof, is used twice a day at a dose of about 80 mg for eight consecutive day.
Embodiment 293. The compound of any one of Embodiments 255 to 257 or any one of Embodiments 288 to 289, wherein Compound (1), or pharmaceutically acceptable salt thereof, is used twice a day at a dose of about 120 mg for eight consecutive day.
Embodiment 294. The compound of any one of Embodiments 255 to 257 or any one of Embodiments 288 to 289, wherein Compound (1), or pharmaceutically acceptable salt thereof, is used twice a day at a dose of about 150 mg for eight consecutive day.
Embodiment 295. The compound of any one of Embodiments 255 to 257 or any one of Embodiments 288 to 289, wherein Compound (1), or pharmaceutically acceptable salt thereof, is used twice a day at a dose of about 300 mg for eight consecutive day.
Embodiment 296. The compound of any one of Embodiments 255 to 295, wherein PRC2-mediated disease or disorder is diffused large B cell lymphoma (DLBCL), follicular lymphoma, leukemia, multiple myeloma, mesothelioma, gastric cancer, malignant rhabdoid tumor, hepatocellular carcinoma, prostate cancer, breast carcinoma, bile duct cancer, gallbladder cancers, bladder carcinoma, neuroblastoma, schwannoma, glioma, glioblastoma, astrocytoma, cervical cancer, colon cancer, melanoma, endometrial cancer, esophageal cancer, head and neck cancer, lung cancer, nasopharyngeal carcinoma, ovarian cancer, pancreatic cancer, renal cell carcinoma, rectal cancer, thyroid cancer, parathyroid tumor, uterine tumor, or soft tissue sarcoma.
Embodiment 297. The method of any one of Embodiments 255 to 296, wherein the PRC2-mediated disease or disorder is diffused large B cell lymphoma (DLBCL), gastric cancer, prostate cancer, nasopharyngeal carcinoma, ovarian cancer, or soft tissue sarcoma.
Embodiment 298. Compound (1), or a pharmaceutically acceptable salt thereof, for use in the treatment of a PRC2-mediated disease or disorder, wherein Compound (1),or a pharmaceutically acceptable salt thereof, is used once a day in an amount sufficient to provide Compound (1) at a dose of about 10 mg to about 800 mg, or Compound (1), or a pharmaceutically acceptable salt thereof is used twice a day in an amount sufficient to provide Compound (1) at a dose of about 60 mg to about 300 mg.
Embodiment 299. The compound of Embodiment 298, wherein Compound (1), or a pharmaceutically acceptable salt thereof, is used once a day in an amount sufficient to provide Compound (1) at a dose of about 10 mg to about 800 mg.
Embodiment 300. The compound of Embodiment 298 or Embodiment 299, wherein Compound (1), or pharmaceutically acceptable salt thereof, is used once a day in an amount sufficient to provide Compound (1) at a dose of about 10 mg, about 20 mg, about 40 mg, about 80 mg, about 120 mg, about 160 mg, about 240 mg, about 300 mg, about 500 mg or about 800 mg.
Embodiment 301. The compound of any one of Embodiments 298 to 299, wherein Compound (1), or pharmaceutically acceptable salt thereof, is used once a day in an amount sufficient to provide Compound (1) at a dose of about 10 mg.
Embodiment 302. The compound of any one of Embodiments 298 to 299, wherein Compound (1), or pharmaceutically acceptable salt thereof, is used once a day in an amount sufficient to provide Compound (1) at a dose of about 20 mg.
Embodiment 303. The compound of any one of Embodiments 298 to 299, wherein Compound (1), or pharmaceutically acceptable salt thereof, is used once a day in an amount sufficient to provide Compound (1) at a dose of about 40 mg.
Embodiment 304. The compound of any one of Embodiments 298 to 299, wherein Compound (1), or pharmaceutically acceptable salt thereof, is used once a day in an amount sufficient to provide Compound (1) at a dose of about 80 mg.
Embodiment 305. The compound of any one of Embodiments 298 to 299, wherein Compound (1), or pharmaceutically acceptable salt thereof, is used once a day in an amount sufficient to provide Compound (1) at a dose of about 120 mg.
Embodiment 306. The compound of any one of Embodiments 298 to 299, wherein Compound (1), or pharmaceutically acceptable salt thereof, is used once a day in an amount sufficient to provide Compound (1) at a dose of about 160 mg.
Embodiment 307. The compound of any one of Embodiments 298 to 299, wherein Compound (1), or pharmaceutically acceptable salt thereof, is used once a day in an amount sufficient to provide Compound (1) at a dose of about 240 mg.
Embodiment 308. The compound of any one of Embodiments 298 to 299, wherein Compound (1), or pharmaceutically acceptable salt thereof, is used once a day in an amount sufficient to provide Compound (1) at a dose of about 300 mg.
Embodiment 309. The compound of any one of Embodiments 298 to 299, wherein Compound (1), or pharmaceutically acceptable salt thereof, is used once a day in an amount sufficient to provide Compound (1) at a dose of about 500 mg.
Embodiment 310. The compound of any one of Embodiments 298 to 299, wherein Compound (1), or pharmaceutically acceptable salt thereof, is used once a day in an amount sufficient to provide Compound (1) at a dose of about 800 mg.
Embodiment 311. The compound of embodiment 298, wherein Compound (1), or a pharmaceutically acceptable salt thereof, is used twice a day in an amount sufficient to provide Compound (1) at a dose of about 60 mg to about 300 mg.
Embodiment 312. The compound of Embodiment 298 or Embodiment 311, wherein Compound (1), or pharmaceutically acceptable salt thereof, is used twice a day in an amount sufficient to provide Compound (1) at a dose of about 60 mg, about 80 mg, about 120 mg, about 150 mg, or about 300 mg.
Embodiment 313. The compound of Embodiment 298 or any one of Embodiments 311 to 312, wherein Compound (1), or pharmaceutically acceptable salt thereof, is used twice a day in an amount sufficient to provide Compound (1) at a dose of about 60 mg.
Embodiment 314. The compound of Embodiment 298 or any one of Embodiments 311 to 312, wherein Compound (1), or pharmaceutically acceptable salt thereof, is used twice a day in an amount sufficient to provide Compound (1) at a dose of about 80 mg.
Embodiment 315. The compound of Embodiment 298 or any one of Embodiments 311 to 312, wherein Compound (1), or pharmaceutically acceptable salt thereof, is used twice a day in an amount sufficient to provide Compound (1) at a dose of about 120 mg.
Embodiment 316. The compound of Embodiment 298 or any one of Embodiments 311 to 312, wherein Compound (1), or pharmaceutically acceptable salt thereof, is used twice a day in an amount sufficient to provide Compound (1) at a dose of about 150 mg.
Embodiment 317. The compound of Embodiment 298 or any one of Embodiments 311 to 312, wherein Compound (1), or pharmaceutically acceptable salt thereof, is used twice a day in an amount sufficient to provide Compound (1) at a dose of about 300 mg.
Embodiment 318. The compound of any one of Embodiments 298 to 317, wherein PRC2-mediated disease or disorder is diffused large B cell lymphoma (DLBCL), follicular lymphoma, leukemia, multiple myeloma, mesothelioma, gastric cancer, malignant rhabdoid tumor, hepatocellular carcinoma, prostate cancer, breast carcinoma, bile duct cancer, gallbladder cancers, bladder carcinoma, neuroblastoma, schwannoma, glioma, glioblastoma, astrocytoma, cervical cancer, colon cancer, melanoma, endometrial cancer, esophageal cancer, head and neck cancer, lung cancer, nasopharyngeal carcinoma, ovarian cancer, pancreatic cancer, renal cell carcinoma, rectal cancer, thyroid cancer, parathyroid tumor, uterine tumor, or soft tissue sarcoma.
Embodiment 319. The method of any one of Embodiments 298 to 318, wherein the PRC2-mediated disease or disorder is diffused large B cell lymphoma (DLBCL), gastric cancer, prostate cancer, nasopharyngeal carcinoma, ovarian cancer, or soft tissue sarcoma.

### EXAMPLES

### Example 1: A Phase I/II, Multicenter, Open-Label Study of MAK683 in Adult Subjects With Advanced Malignancies

This study is a multicenter, open label, study to establish the maximum tolerated dose (MTD) and/or recommended phase 2 dose (RP2D) and to evaluate the safety, antitumor activity and pharmacokinetic (PK) profile of MAK683 (Compound (1) of MAK683 (Compound (1) in subjects with advanced malignancies such as Diffuse Large B cell Lymphoma (DLBCL), nasopharyngeal carcinoma (NPC) or other advanced solid tumors for whom no further effective standard treatment is available. The patients will be administered MAK683 single agent orally until they experience unacceptable toxicity, progressive disease and/or treatment discontinuation at the discretion of the investigator or patient or withdrawal of consent. The phase I part plans to enroll patients with relapsed/refractory DLBCL, advanced/ metastatic nasopharyngeal carcinoma with presence of p16/CDKN2A gene and other solid tumors including castration-resistant prostate cancer, gastric cancer, OCCC, sarcoma. All patients enrolled in this part of the study will receive escalating oral doses of MAK683 in fasted condition, unless significant GI toxicity is observed, in which case dosing with moderate-fat meals may be assessed. The starting dose and regimen for the MAK683 first-in-man clinical study is about 10 mg QD. Escalation will continue to about 20 mg, about 40 mg, about 80 mg, about 120 mg, about 160 mg, about 160 mg, about 240 mg, about 300 mg, about 500 mg or about 800 mg or until the MTD is reached or a RP2D is established based on emerging data.

Once the MTD and/or RP2D have been declared, additional patients will be enrolled in the phase II part in order to assess the preliminary anti-tumor activity of MAK683. Approximately 100 patients will be enrolled in the phase II part, consisting of 4 groups. Group 1 will enroll approximately 20 relapsed/refractory DLBCL patients with EZH2 mutations confirmed centrally at a designated laboratory. Group 2 will enroll approximately 20 relapsed/refractory DLBCL patients with no EZH2 mutations confirmed centrally at a designated laboratory. Group 3 will enroll approximately 20 patients with advanced/metastatic nasopharyngeal carcinoma with presence of p16/CDKN2A gene confirmed centrally at a designated laboratory. Group 4 will enroll patients with advanced/metastatic gastric cancer, castration-resistant prostate cancer, OCCC and sarcoma characterized by SWI/SNF alterations, approximately 40 patients will be enrolled in this group with an aim to have approximately 10 patients for each indication. Should more than one dose be identified for further investigation during the phase II part, then these dose(s) will be tested in one or more indications to better assess the safety, benefit-risk and anti-tumor activity of MAK683 based in part on logistical feasibility. In this case, the dose levels would be assigned in an alternating fashion to patients of the same disease group across all the sites in this global study. A treatment cycle is defined as 28 days for the purposes of scheduling procedures and evaluations.

A positive endpoint is met if treatment results in an improved overall response rate (ORR), based on local assessment per response assessment of Hodgkin and Non-Hodgkin Lymphoma (Cheson et al (2014) J Clin Oncol; 32(27): 3059-68), Response Evaluation Criteria in Solid Tumors (RECIST) v1.1 and PCWG2. The study results showed that MAK683 met at least one of the above endpoints and any adverse events were mild to moderate and did not lead to discontinuation.

MAK683 was administered fasted once (QD) or twice daily (BID) on a continuous schedule in 28-day treatment cycles. The pharmacokinetic (PK) profile of MAK683 was assessed in sequential blood samples on Days 1, 8 and/or 15 of Cycles 1-6. MAK683 pharmacodynamic activity in Cycle 1, measured by change in H3K27me3, was evaluated in peripheral blood monocytes on Days 1, 8, and 15 by flow cytometry and in tumor biopsies at baseline and Day 15 by H-score.

As of Jan. 15, 2021, 125 patients had received MAK683 at doses of 10-800 mg QD or 60-450 mg BID. MAK683 was rapidly absorbed with a median Tₘₐₓ of ˜1 hour. PK exposure (Cₘₐₓ and AUC) was variable and broadly dose-proportional for 10-500 mg QD and 60-450 mg BID but over-proportional at 800 mg QD. Apparent terminal half-life (geometric mean) was 2.5-5.2 hours across cohorts and unchanged after multiple doses. MAK683 accumulation was noted after multiple doses (R_{acc} 0.8-2.3). Time to steady-state was ˜3 days. Peripheral monocytes showed substantial on-treatment reductions from baseline in the H3K27me3/H3 ratio across doses. Maximum percentage reduction was proportional to cumulative MAK683 AUC, with a trend towards greater reductions at higher baseline H3K27me3. H3K27me3 H-score reductions from baseline >40 were observed in 7/10 patients with diffuse large B-cell lymphoma (n=4) or sarcoma (n=6) and paired baseline-Day 15 biopsies. These results show that MAK683 has a PK profile supportive of QD or BID dosing in patients with advanced malignancies. In addition, the analysis of H3K27me3 in blood monocytes and tumor biopsy confirm the in vivo pharmacodynamic activity of MAK683.

Unless defined otherwise, the technical and scientific terms used herein have the same meaning as they usually understood by a specialist familiar with the field to which the disclosure belongs.

Unless indicated otherwise, all methods, steps, techniques and manipulations that are not specifically described in detail can be performed and have been performed in a manner known per se, as will be clear to the skilled person. Reference is for example again made to the standard handbooks and the general background art mentioned herein and to the further references cited therein. Unless indicated otherwise, each of the references cited herein is incorporated in its entirety by reference.

Claims to the invention are non-limiting and are provided below. Although particular aspects and claims have been disclosed herein in detail, this has been done by way of example for purposes of illustration only, and is not intended to be limiting with respect to the scope of the appended claims, or the scope of subject matter of claims of any corresponding future application. In particular, it is contemplated by the inventors that various substitutions, alterations, and modifications may be made to the disclosure without departing from the spirit and scope of the disclosure as defined by the claims. The choice of nucleic acid starting material, clone of interest, or library type is believed to be a matter of routine for a person of ordinary skill in the art with knowledge of the aspects described herein. Other aspects, advantages, and modifications considered to be within the scope of the following claims. Those skilled in the art will recognize or be able to ascertain, using no more than routine experimentation, many equivalents of the specific aspects of the invention described herein. Such equivalents are intended to be encompassed by the following claims. Redrafting of claim scope in later filed corresponding applications may be due to limitations by the patent laws of various countries and should not be interpreted as giving up subject matter of the claims.

1. A method for treating a PRC2-mediated disease or disorder in a subject in need thereof, comprising administering to said subject a therapeutically effective amount of N-((5-fluoro-2,3-dihydrobenzofuran-4-yl)methyl)-8-(2-methylpyridin-3-yl)-[1,2,4]triazolo[4,3-c]pyrimidin-5-amine (Compound (1)), or a pharmaceutically acceptable salt thereof, 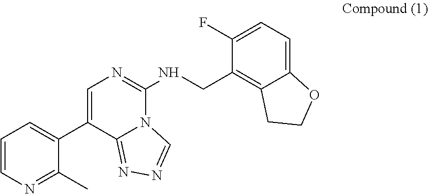 [SMILES: Cc1ncccc1-c1cnc(NCc2c(F)ccc3c2CCO3)n2cnnc12]
wherein Compound (1), or a pharmaceutically acceptable salt thereof, is administered daily.

2. The method of claim 1, wherein Compound (1) is administered as the hydrochloride salt.

3. The method of claim 1 or claim 2, wherein Compound (1), or a pharmaceutically acceptable salt thereof, is administered at least once a day.

4. The method of any one of claims 1 to 3, wherein Compound (1), or a pharmaceutically acceptable salt thereof, is administered at least once a day at a dose of about 10 mg to about 800 mg.

5. The method of any one of claims 1 to 4, wherein Compound (1), or a pharmaceutically acceptable salt thereof, is administered at least once a day at a dose of about 10 mg, about 20 mg, about 40 mg, about 80 mg, about 120 mg, about 160 mg, about 240 mg, about 300 mg, about 500 mg or about 800 mg.

6. The method of claim 1 or claim 2, wherein Compound (1), or a pharmaceutically acceptable salt thereof, is administered twice a day.

7. The method of any one of claim 1, 2 or 6, wherein Compound (1), or a pharmaceutically acceptable salt thereof, is administered twice a day at a dose of about 60 mg to about 300 mg.

8. The method of any one of claims 1, 2, 6 or 7, wherein Compound (1), or a pharmaceutically acceptable salt thereof, is administered twice a day at a dose of about 60 mg, about 80 mg, about 120 mg, about 150 mg, or about 300 mg.

9. The method of any one of claims 1 to 8, wherein Compound (1), or a pharmaceutically acceptable salt thereof, is orally administered.

10. The method of any one of claims 1 to 9, wherein Compound (1), or a pharmaceutically acceptable salt thereof, is administered as a continuous dosing regimen comprising one or more continuous dosing cycles.

11. The method of claim 10, wherein the continuous dosing regimen comprises two continuous dosing cycles.

12. The method of claim 10 or claim 11, wherein each continuous dosing cycle is up to an twenty eight day cycle.

13. The method of any one of claims 1 to 9, wherein Compound (1), or a pharmaceutically acceptable salt thereof, is administered as a cyclical dosing regimen comprising one or more continuous dosing cycles and one or more dose delays.

14. The method of claim 13, wherein the cyclical dosing regimen comprising two continuous dosing cycles and one dose delay.

15. The method of claim 13 or claim 14, wherein each continuous dosing cycle is up to an eight day cycle and the dose delay is eight or more days.

16. The method of any one of claims 1 to 15, wherein the PRC2-mediated disease or disorder is diffused large B cell lymphoma (DLBCL), follicular lymphoma, leukemia, multiple myeloma, mesothelioma, gastric cancer, malignant rhabdoid tumor, hepatocellular carcinoma, prostate cancer, breast carcinoma, bile duct cancer, gallbladder cancers, bladder carcinoma, neuroblastoma, schwannoma, glioma, glioblastoma, astrocytoma, cervical cancer, colon cancer, melanoma, endometrial cancer, esophageal cancer, head and neck cancer, lung cancer, nasopharyngeal carcinoma, ovarian cancer, pancreatic cancer, renal cell carcinoma, rectal cancer, thyroid cancer, parathyroid tumor, uterine tumor, or soft tissue sarcoma.

17. The method of any one of claims 1 to 16, wherein the PRC2-mediated disease or disorder is diffused large B cell lymphoma (DLBCL), gastric cancer, prostate cancer, nasopharyngeal carcinoma, ovarian cancer, or soft tissue sarcoma.
